SEQUENCE LISTING (3 sequences)

SEQ ID NO: 1 - protein, 654 aa, Artificial Sequence
  Synthetic Sequence
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLR

SEQ ID NO: 2 - protein, 1242 aa, Artificial Sequence
  Synthetic sequence
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 421 aa, Artificial Sequence
  synthetic sequence
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQ

(19) United States
(12) Patent Application Publication
(10) Pub. No.: US 2022/0003779 A1
(43) Pub. Date: Jan. 6, 2022

(54) METHODS FOR QUALITATIVE AND QUANTITATIVE ANALYSIS OF A PLURALITY OF BIOMARKERS

(71) Applicant: The Research Foundation for the State University of New York, Albany, NY (US)

(72) Inventor: Nathaniel Cady, Delmar, NY (US)

(21) Appl. No.: 17/367,141

(22) Filed: Jul. 2, 2021

Related U.S. Application Data
(60) Provisional application No. 63/048,001, filed on Jul. 3, 2020.
(60) Provisional application No. 63/215,839, filed on Jun. 28, 2021.

(51) Int. Cl. G01N 33/68; G01N 21/64

(52) U.S. Cl. CPC G01N 33/6854; G01N 21/6428

(57) ABSTRACT

The present disclosure relates to a method for the simultaneous qualitative and quantitative analysis of a plurality of biomarkers contained in a sample, including: capturing one or more biomarkers on a substrate configured to bind a plurality of biomarkers to a plurality of binding sites, wherein binding sites bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light emits light at a predetermined wavelength for transferring energy to surface plasmons and excite fluorescence of the one or more fluorescent complexes; and detecting emission light of fluorescent complexes; and determining the type and/or quantity of plurality of biomarkers.

### CROSS-REFERENCE TO RELATED APPLICATIONS

The present disclosure claims priority or the benefit under 35 U.S.C. § 119 of U.S. provisional application Nos. 63/048,001 filed Jul. 3, 2020, and 63/215,839 filed 28 Jun. 2021, both of which are fully incorporated herein by reference.

### REFERENCE TO A SEQUENCE LISTING

This application contains a Sequence Listing in computer readable form, which is incorporated herein by reference.

### FIELD OF THE INVENTION

The present disclosure relates to apparatuses and methods for detecting the amount and/or type of one or more analytes-of-interests such as biomarkers in a sample. In embodiments, the present disclosure relates to apparatuses and methods for the quantitative detection of biomarkers against an infectious agent or vaccine, such as detecting one or more human antibodies against the SARS-CoV-2 virus, viral variants of SARS-CoV-2, and/or one or more predetermined biomarkers.

### BACKGROUND

Infectious diseases cause acute and chronic health problems in humans, whether due to pathogens such as bacteria, fungi, viruses, or other factors. Infections occur when pathogens enter the body and multiply. Disease occurs when the cells of the body are damaged by the infection, and signs and symptoms of illness appear.

Infectious diseases continue to develop naturally resulting from new pathogens and strains infecting human populations. For example, in the mid-1970's Lyme disease caused by ticks infected with the spirochete bacteria, Borrelia burgdorferi, was identified in rural Connecticut causing symptoms similar to rheumatoid arthritis. More recently the World Health Organization (WHO) declared infection by the novel Severe Acute Respiratory Syndrome Coronavirus 2 (SARS-CoV-2), as a pandemic, and termed the related disease as coronavirus disease 2019 (COVID-19). Variants of interest of SARS-CoV-2 and variants of concern of SARS-CoV-2 have been identified, and it is expected that additional variants will present as the virus continues to evolve over time.

Upon infection, the human body senses foreign substances or antigens and the immune system works to recognize antigens and rid the body of the antigens. Adaptive immunity develops when individuals are exposed to antigens and typically becomes more prominent after several days of infection, as antigen-specific T and B cells have undergone clonal expansion. For a general overview of the human immune system response, see for example, Chaplin, Overview of the Immune Response, J Allergy Clin Immunol. 2010 February; 125(2 Suppl 2): S3-23 (herein entirely incorporated by reference). A typical immune response may include the production of biomarkers such as immunological agents or immunoglobulins in various isoforms including IgG, IgA, IgM and IgE antibodies, and by somatic mutations in the antigen-binding domains of the heavy and light chains of these antibodies.

The inventor has observed that the quantity and type of biomarkers such as immunological agents may vary over the course of infection, disease, and recovery, and that biomarkers may be monitored to determine a subject's status. Such monitoring may be useful for disease intervention or managing a treatment strategy. Further, monitoring biomarkers such as antibodies throughout a subject's recovery period is problematic in that it typically requires multiple pieces of laboratory equipment in a complex laboratory environment to quantify and type the biomarkers, especially where the biomarkers may be present at low concentrations or where sample quality is poor. Moreover, the form of the biological samples may present problems depending upon whether the sample is serum, whole blood, or dried blood, as varying conditions of the sample may make evaluation thereof difficult.

Further, the inventor has found that detection of a virus-of-interest alone has several shortcomings in treatment management. For example, one currently available diagnostic test, a PCR based approach for COVID-19 testing has several deficiencies, namely the test only detects the presence of virus in the sample and does not determine the immune status of one or more subjects, especially where subjects have different immune responses to an infection.

The inventor has also observed that medical doctors or clinicians would benefit from monitoring a subject's status and the immune response over the duration of an illness or recovery period.

Accordingly, there is a need for improved methods, apparatuses, and assays for the detection and identification of one or more biomarkers such as those made in response to viral diseases, bacterial diseases, or other diseases. What is needed are methods of simultaneously detecting and/or quantifying biomarkers such as antibodies over the course of treatment, such as from a blood, blood serum, or dried blood. Moreover, what is also needed are methods of surveilling a subject's immune status to estimate or determine the type of immunological response to an infection.

### SUMMARY

The present disclosure relates to methods for detecting and/or quantifying biomarkers in a specimen, such as from a blood, blood serum, or dried blood. In embodiments, the present disclosure relates to a method for a simultaneous qualitative and quantitative analysis of a plurality of biomarkers contained in a sample, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

In some embodiments, the present disclosure relates to an apparatus for qualitative and quantitative analysis of a plurality of biomarkers contained in a sample, including: a substrate including a plurality of binding sites, wherein two or more of the plurality of binding sites include two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, bind to one or more fluorescent binding partners to form one or more fluorescent complexes; and a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes.

In some embodiments, the present disclosure relates to a non-transient computer readable medium having instructions stored thereon that, when executed, causes an apparatus for qualitative and quantitative analysis of a plurality of biomarkers contained in a sample to perform a method, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the computer readable media is non-transitory computer readable media.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flowchart of a method for a simultaneous qualitative and quantitative analysis of a plurality of biomarkers in accordance with some embodiments of the present disclosure.

FIGS. 2A-2D are illustrative cross-sectional views of the substrate during different stages of the process sequence of FIG. 1 in accordance with some embodiments of the present disclosure.

FIG. 3 depicts a tool suitable for processing a substrate in accordance with some embodiments of the present disclosure.

FIG. 4 is a photograph of a microfluidic flow cell such as GC-FP biochip suitable for use with some embodiments of the present disclosure.

FIG. 5 is a photograph of a fluorescent image in accordance with the present disclosure.

FIG. 6A is a map of antigen spots in accordance with the present disclosure.

FIG. 6B is an image of antigen spots of FIG. 6A processed in accordance with the present disclosure.

FIG. 7A is a plot of GC-FP intensity in accordance with one embodiment of the present disclosure.

FIG. 7B is a plot showing the scoring of the plot shown in FIG. 7A.

FIGS. 8A and 8B are photographs of a dipstick and holder in accordance with embodiments of the present disclosure.

FIG. 9 is a photograph of six 4×4 GC-FP chips with 3×3 grid of binding spots in accordance with the present disclosure.

FIG. 10 shows an image of serum and blood test using the kit shown in FIG. 9.

FIGS. 11A and 11B show graphs and images of dried blood samples analyzed in accordance with the present disclosure.

FIG. 12 show graphs and images of samples analyzed in accordance with the present disclosure.

FIG. 13A depicts a GC-FP biosensor chip of the present disclosure.

FIG. 13B depicts COVID-19 antigens or control proteins spotted onto GC-FP biosensor chips, then assessed for antibody binding from human blood samples.

FIG. 13C depicts fluorescence images of GC-FP biosensor chips of the present disclosure.

FIGS. 14A and 14B depict GC-FP results for serum (FIG. 14A) and dried blood spot (FIG. 14B) samples from subjects with verified positive COVID-19 infection status.

FIGS. 14C and 14D depict plots of data obtained in accordance with the present disclosure.

FIG. 15A-15C depict visualization of testing data following machine learning-based analysis of the present disclosure.

FIGS. 16A-16D depict quantitative comparison of GC-FP and ELISA for detection of IgG against multiple COVID-19 antigens.

FIGS. 17A-17B depict serum sample tested on three separate GC-FP biosensor chips that included RBD, S1, S1S2, and Nuc antigens.

FIG. 18 depicts various GC-FP biosensor chips of the present disclosure and photographs of observable results.

FIGS. 19A and 19B depict receiver operator characteristic (ROC) analysis for serum (FIG. 19A) and dried blood spot (FIG. 19B) samples with known COVID-19 infection history.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The apparatuses and methods described herein relate to detecting and/or quantifying biomarkers-of-interest, such as from infected or diseased subjects. In embodiments, apparatuses and methods for detecting and/or quantifying biomarkers in a specimen, such as from whole blood, blood serum, or dried blood are described.

In embodiments, the present disclosure relates to a method for a simultaneous qualitative and quantitative analysis of a plurality of biomarkers contained in a sample, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

Embodiments of present disclosure advantageously provide: improved methods, apparatuses, and assays for the detection and identification of one or more biomarkers such as those made in response to viral diseases, bacterial diseases or other diseases; methods that do not require complex laboratory infrastructure and can be applied to any infectious disease that elicits an immune response, or where antibody levels need to be determined for diagnosis/prognosis; methods and apparatuses for detecting and/or simultaneously quantifying biomarkers such as antibodies over the course of treatment, such as from a blood, blood serum, or dried blood; or methods of surveilling a subject's immune status to estimate or determine type of immunological response to an infection. Additional benefits of the methods and apparatuses of the present disclosure may include providing a comprehensive diagnostic strategy that in a single test allows for simultaneous detection of antibodies; cytokines; and other biomarkers essential in properly detecting pathogen infection such as from the bacteria or virus such as SARS-CoV-2 and the severity of infection in each subject. Advantages may be especially important where convalescent subjects such as COVID-19 patients are being screened for use of their serum/blood sample to provide to infected patients for therapy.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the term “ACE2” refers to Angiotensin II converting enzyme (ACE2, EC 3.4.17.23). ACE2 is a protein that catalyzes the cleavage of angiotensin I into angiotensin 1-9, and angiotensin II into the vasodilator angiotensin 1-7. In addition, the encoded protein is a functional receptor for the spike glycoprotein of the human coronavirus HCoV-NL63 and the human severe acute respiratory syndrome coronaviruses, SARS-CoV and SARS-CoV-2 (COVID-19 virus). ACE2 converting enzyme activity may be measured using, inter alia, fluorometric activity assay kits and by other known methods such as those described in Measurement of Angiotensin Converting Enzyme 2 Activity in Biological Fluid (ACE2), Methods Mol Biol. 2017; 1527:101-115.

The term “antibody” as used herein refers to an immunoglobulin molecule capable of specific binding to a target antigen or biomarker, such as a carbohydrate, polynucleotide, lipid, polypeptide, peptide etc., via at least one antigen recognition site (also referred to as a binding site), located in the variable region of the immunoglobulin molecule.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

As used herein, the terms “bind” and “binding” generally refer to the non-covalent interaction between a pair of partner molecules or portions thereof (e.g., antigenic protein-binding partner complexes) that exhibit mutual affinity or binding capacity. In embodiments, binding can occur such that the partners are able to interact with each other to a substantially higher degree than with other, similar substances. This specificity can result in stable complexes (e.g., antigenic protein-binding partner complexes or bound biomarkers-of-interest) that remain bound during handling steps such as chromatography, centrifugation, filtration, and other techniques typically used for separations and other processes. In embodiments, the interaction between a target region of an antigenic protein and a binding partner that binds specifically thereto is a non-covalent interaction. In some instances, the interaction between a binding partner and a non-target region of an antigenic protein is a non-covalent interaction. However, in other instances, the interaction between a binding partner and a non-target region of an antigenic protein may be a covalent interaction. In embodiments, a protein complex comprising the antigenic protein and the binding partner may be contacted with a chemical crosslinking reagent that causes covalent bonds between the antigenic protein and the binding partner to be formed. In another example, the antigenic protein may contain a first reactive chemical moiety (handle) and the one or more binding partners may each contain a second reactive chemical moiety (handle), wherein the first and second chemical reactive moieties can react with each other to form a covalent bond. Exemplary reactive chemical moieties include those useable in “click” chemistry, which is a class of biocompatible small molecule reactions commonly used in bioconjugation, allowing the joining of substrates of choice with specific biomolecules. Click chemistry is not a single specific reaction, but refers to a way of generating products that follow examples in nature, which also generates substances by joining small modular units. In one example, the antigenic protein may have a first reactive chemical moiety such as a clickable handle like an azide, and the binding partner(s) could have a complementary reactive handle such as, for example a strained cyclooctyne, or vice versa. When these reactive chemical moieties come into proximity when the antigenic protein and the one or binding partners interact to form a protein complex, they can react with each other to form a covalently bond between the proteins.

“Biological sample” refers to isolation of tissue and/or fluid from a subject that is being tested for a disease state. In embodiments, any biological sample can be used by the present disclosure, provided the sample may contain, or contains the biomarkers for the disease state being tested, such as blood, blood components, urine, saliva or breath. In embodiments, biological samples include blood, blood plasma, or dried blood.

“Biomarker” refers to biological compounds that are involved in one or more biological pathways that are associated with a disease state. Accordingly, for infections, the biomarker can be involved with pathways that regulate the host immune response. A “profile” of biomarkers or “biomarker profile” refers to the amount or concentration of two or more biomarkers. Such a profile provides useful top-level “fluxomics” information about whether certain types or pools of biomarkers are elevated or depleted. A disease state can have a specific biomarker profile, and more particularly a time-dependent biomarker profile. Accordingly, the biomarker profile is also referred to as a disease state “finger-print” that permits the identification of a disease state based on a measured biomarker profile. A biomarker that is “related to the disease state” refers to biomarker profiles that change depending on the disease state and provides a means for assessing a subject's disease state based on the measured biomarker levels.

The terms “elute” and “eluting” refer to the disruption of non-covalent interactions between partner molecules (e.g., a modified antigenic protein and a binding partner) such that the partners become unbound from one another. The disruption can be effected via introduction of a competitive binding species, or via a change in environmental conditions (e.g., ionic strength, pH, or other conditions).

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

As used herein the terms “dye” and “label” are used interchangeably to designate fluorescent molecules.

The term “fluorescent complexes” as used herein means complexes such as biomolecules, proteins, protein conjugates and the like that can fluoresce.

The term “fluoresce” as used herein means to exhibit or undergo the phenomenon of fluorescence.

The term “fluorescence” as used herein means the emission of light by a substance that has absorbed light or other electromagnetic radiation of a different wavelength.

The term “fluoresced light” as used herein means emitted light from fluorescence of a substance. In most cases, emitted light has a longer wavelength, and therefore lower energy, than the absorbed radiation. However, when the absorbed electromagnetic radiation is intense, it is possible for one electron to absorb two photons; this two-photon absorption can lead to emission of radiation having a shorter wavelength than the absorbed radiation.

The term “isolated” refers to a chemical or biomolecule species such as a protein, protein complex, or DNA sequence that is removed from at least one component with which it is naturally associated.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used here, the term “SARS-CoV-2” refers to virus classified within the genus Betacoronavirus (subgenus Sarbecovirus) in the family Coronaviridae (subfamily Orthocoronavirinae), a family of single-strand positive-sense RNA viruses. In embodiments, the term “SARS-CoV-2” includes variants of SARS-CoV-2.

The term “solid support” is used herein to denote a solid inert surface or body to which an agent, such as a binding partner, that is reactive in any of the binding reactions described herein can be immobilized. The term “immobilized” as used herein denotes a molecularly-based coupling that is not dislodged or de-coupled under any of the conditions imposed during any of the steps of the assays described herein. Such immobilization can be achieved through a covalent bond, an ionic bond, an affinity-type bond, or any other covalent or non-covalent bond. Exemplary solid supports include chromatography resins and multi-well plates, or the substrate of the present disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF CERTAIN EMBODIMENTS

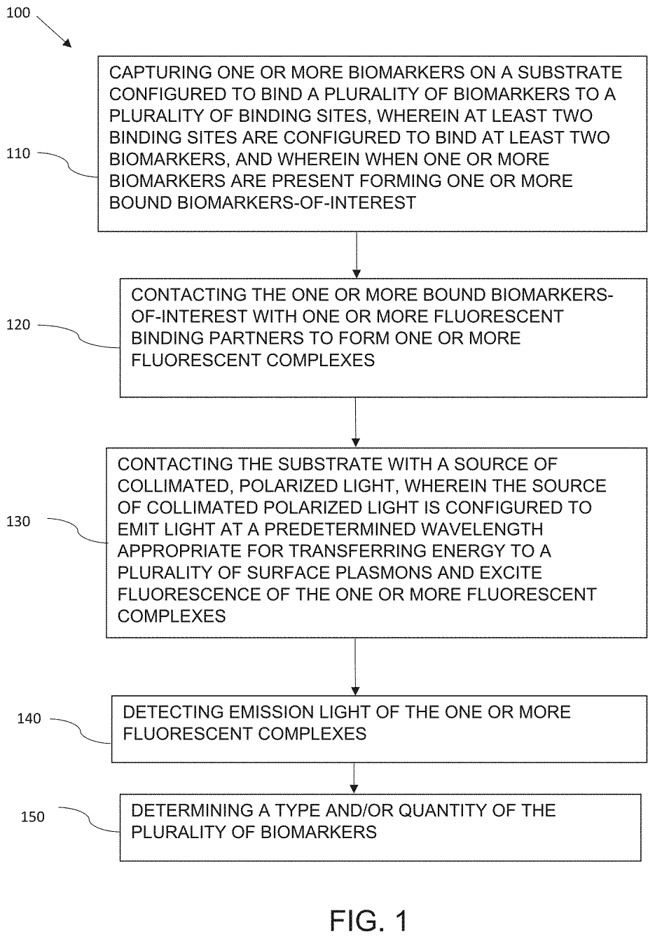

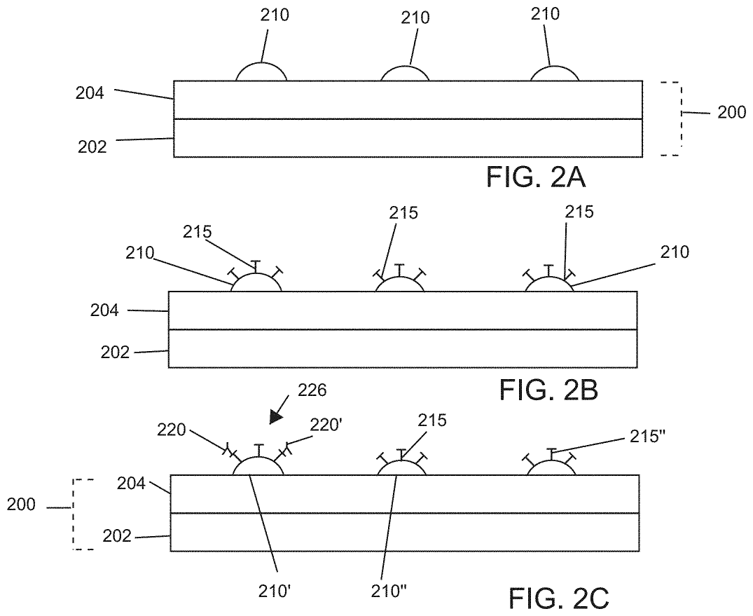

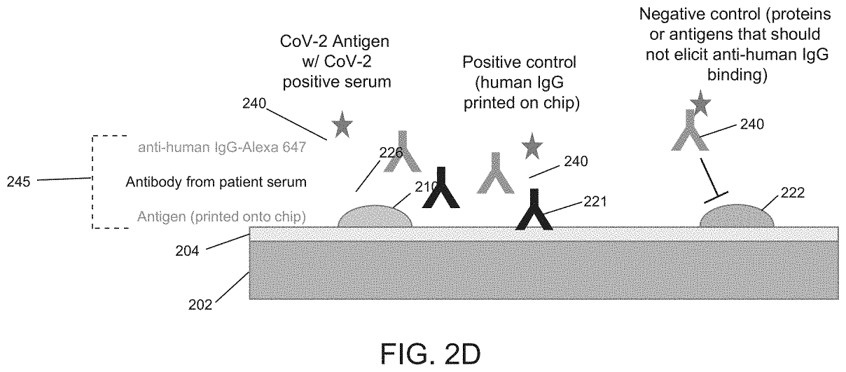

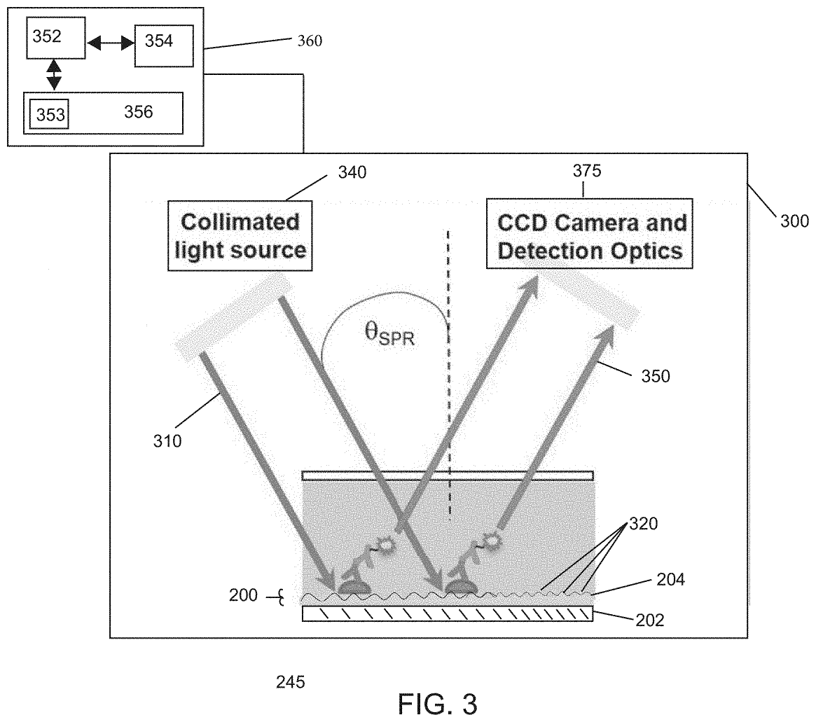

FIG. 1 is a flow diagram of a method 100 for a simultaneous qualitative and quantitative analysis of a plurality of biomarkers contained in a sample in accordance with some embodiments of the present disclosure. The method 100 is described below with respect to the stages of processing a substrate as depicted in FIGS. 2A-2D and may be performed, for example, in a suitable tool, such as grating-coupled fluorescent plasmonic (GC-FP) detection platform described below with respect to FIG. 3. Exemplary processing systems that may be used to perform the methods disclosed herein may include, but are not limited to, any of the GC-FP detection platforms, commercially available from Ciencia, Inc., of East Hartford, Conn. Other GC-FP detection platforms, including ones available from other manufacturers, may also be suitably used in connection with the teachings provided herein.

The method 100 is typically performed on a substrate 200 provided to a grating-coupled fluorescent plasmonic (GC-FP) detection platform described below with respect to FIG. 3. In some embodiments, as shown in FIG. 2A, the substrate 200 includes one or more layers such as first layer 202 and second layer 204 disposed atop the first layer 202. As shown in FIG. 2A, a plurality of, or one or more binding sites 210 are disposed atop the second layer 204. Although the following description is made with respect to three binding sites of the one or more binding sites 210 as shown, the substrate 200 may include any number of the one or more binding sites 210 configured to bind one or more (several) biomarkers as described below. In embodiments, binding sites may be configured as a positive or negative control depending upon experimental design needs, if any. In embodiments, the one or more binding sites 210 include one or more, or several antigens preselected to bind to a biomarker-of-interest.

In embodiments, the substrate 200 may include a first layer 202 including one or more of silicon (Si), silicon oxide (SiO₂), polymer, or the like. In embodiments, the substrate 200 is not limited to any particular size or shape. The substrate 200 can be a round wafer having a 100 mm diameter, 150 mm diameter, 200 mm diameter, a 300 mm diameter or other diameters, such as 450 mm, among others. The substrate can also be any polygonal, square, rectangular, curved or otherwise non-circular workpiece. In some embodiments, as described further below, the substrate 200 is sized to fit within a test-tube.

In embodiments, substrate 200 includes a second layer 204 disposed atop the first layer 202. In embodiments, the second layer 204 is configured for use in a grating-coupled fluorescent plasmonic (GC-FP) detection platform and may include undulations of the first layer 202 and one or more metals such as a metal film including gold, silver or the like in a thickness sufficient to cover the first layer 202 such as 1 to 100 nanometers, such as about 50 nanometers. In some embodiments, second layer 204 is a gold layer disposed atop the first layer 202. In embodiments, as shown in FIG. 3, the second layer 204 may have a plurality of undulations 320 having a plurality of peaks and troughs in a predetermined depth and position. In embodiments, the substrate 200 includes additional layers of materials or may have one or more completed or partially completed one or more binding sites 210 such as a plurality of binding sites disposed upon, formed in, or printed atop the substrate 200 such as directly atop the second layer 204 using any suitable deposition process known in the art. In some embodiments, the one or more binding sites 210 are printed directly atop second layer 204 and affixed atop second layer 204. In some embodiments, printing is performed as described in U.S. Pat. No. 9,400,353 to Cunningham et al. (herein entirely incorporated by reference). In some embodiments, the one or more binding sites 210 include one or more, several (3 to 6), or a plurality of binding partners such as antigens capable of undergoing a binding reaction with a biomarker to be analyzed such as a biomarker-of-interest. Biomolecule complex formation, antigen-antibody binding, or protein-protein binding is understood in the art. See e.g., U.S. patent publication nos. 2021/0139542 and 2019/0322739 (both of which are herein incorporated entirely by reference) for additional information relating to biomolecule binding interactions.

In embodiments, the one or more binding sites 210 are configured to bind a plurality of biomarkers to a plurality of binding sites, wherein at least two binding sites are configured to bind at least two biomarkers, and wherein when one or more biomarkers are present forming one or more bound biomarkers-of-interest. In embodiments, the biomarkers may be any biomarker. Biomarkers are understood in the art (See e.g., U.S. Pat. No. 8,026,049 (herein incorporated by reference). Referring to FIGS. 2B and 2C, the one or more binding sites 210 may include one or more, several, or a plurality of antigens 215, 215″ or antibodies capable of undergoing an antigen-antibody reaction with one or more biomarkers to be analyzed such as a biomarker-of-interest contained in a subject's sample or specimen. As a result of the one or more binding sites 210 being fixed on the top surface of the second layer 204, the biomarkers-of-interest 220 combine with the substrate 200 as shown in FIG. 2C. In embodiments, as shown in FIGS. 2C and 2D, fluorescent material 240 such as a dye, label or other predetermined fluorescent molecules may be applied to bind specifically to biomarker-of-interest 220.

In such embodiments, substrate 200 may be configured as a sensor film. In embodiments, substrate 200 is configured for high sensitivity to one or more biomarkers-of-interest 220, and the rate of the biomarker-binding partner reaction can be kept high. In some embodiments, the biomarker-of-interest 220 in a sample is to be detected by an antigen-antibody reaction, e.g., where an antigen (or an antibody) is fixed in or printed atop a film such a metal film and an antibody (or an antigen) within the sample or specimen is to be detected, the antigen-antibody reaction may be promoted by preselecting the positioning of the one or more binding sites 210 atop second layer 204. In some embodiments, a plurality of biomarkers-of-interest such as 1 to 50, 1 to 30, 1 to 15, or 1 to 10 biomarkers-of-interest in a sample may be detected by antigen-antibody reactions, e.g., where a plurality of antigens (or antibodies) such as 1 to 50, 1 to 30, 1 to 15, or 1 to 10 antigens are fixed in a metal film and a plurality of biomarkers such as a plurality of antibodies (or antigens) such as 1 to 50, 1 to 30, 1 to 15, or 1 to 10 antibodies within the sample or specimen are to be detected, and multiple (such as 1 to 50, 1 to 30, 1 to 15, or 1 to 10) antigen-antibody reactions may be promoted atop one or more binding sites 210 atop second layer 204. As shown in FIG. 2D, a positive control binding partner 221 may also be printed onto second layer 204, wherein the positive control binding partner 221 is configured to bind to fluorescent material 240. As shown in FIG. 2D, a negative control binding partner 222 may also be printed onto second layer 204, wherein the negative control binding partner 222 is configured not to bind, or does not bind, to fluorescent material 240.

In some embodiments, substrate 200 is configured to bind a plurality of biomarkers to a plurality of the one or more binding sites 210, wherein at least two binding sites (210′ and 210″ of FIG. 2C) of the one or more binding sites 210 are configured to bind at least two biomarkers when present, and wherein when one or more biomarkers are present forming one or more bound biomarkers-of-interest (See FIG. 2B, where biomarker 220 and biomarker 200′ are bound to binding site 210′). In embodiments, several or a plurality of bound-biomarkers-of-interest such as biomarker 220 and biomarker 220′ may be typed and quantified.

Non-limiting examples of suitable biomarkers according to the present disclosure include proteins-of-interest such as proteins for which expression is increased in a subject and have the potential to serve as informative indicators relating to disease or immunological status. In some embodiments, biomarkers refer to any measurable factor that differentiates a normal biological process from a disease related process or its response to therapy. In some embodiments, biomarkers may have a high diagnostic or prognostic performance. In some embodiments, biomarkers are proteins such as antibodies, cytokines, oligonucleotides specific to a target, or fragments thereof. Non-limiting examples of biomarkers include immunoglobulins such as one or more of immunoglobulin G (IgG), immunoglobulin M (IgM), immunoglobulin A (IgA), immunoglobulin (E), or isotypes or combinations thereof. In some embodiments, biomarkers include antibodies or fragments thereof selected from the group consisting of scFv, Fab, and a binding domain of an immunoglobulin molecule. In some embodiments, biomarkers provide diagnostic or prognostic indication of a specific disease. In some embodiments, biomarkers include biomarkers relating to Lyme Disease (LD) such as one or more antigens including: Borrelia burgdorferi outer surface protein (BBA69), plasminogen-binding protein (BBA70), laminin-binding protein (BmpA), Decorin binding proteins A and B (DbpA, DbpB), outer surface protein (ErpL), outer surface protein C (OspC), outer surface protein D (OspD), protein (P41), protein (P58), major protein (VMP)-like sequence (VIs) E lipoprotein (VIsE), or combinations thereof, see for example, Chou et al., A fluorescent plasmonic biochip assay for multiplex screening of diagnostic serum antibody targets in human Lyme disease, PLoS One. 2020; 15(2): e0228772 (herein entirely incorporated by reference).

In some embodiments, the one or more biomarkers include antibodies specific to SARS-CoV-2 or fragments thereof, or variants thereof. In some embodiments, biomarkers are characterized as serum antibody targets as known to one of ordinary skill in the art. In embodiments, antigens suitable for detection of COVID-19 related biomarkers using methods of the present disclosure include SARS-CoV-2 spike protein such as S1 (SEQ ID NO: 1) and fragments thereof such as the receptor binding domain (RBD); SARS-CoV-2 spike protein such as a protein characterized as S1S2 (SEQ ID NO: 2); or SARS-CoV-2 envelope protein such as N (SEQ ID NO: 3). In embodiments, biomarkers may include highly related sequences to these sequences such as polypeptides having at least 90%, 95%, 99% sequence identity to SEQ ID NO:1, SEQ ID NO: 2 or SEQ ID NO: 3. In embodiments, other proteins from SARS-CoV-2, and/or fragments thereof, or variants thereof may be antigens suitable for use in detecting SARS-CoV-2 related biomarkers-of-interest in accordance with the present disclosure. In embodiments, these antigens are disposed upon and bound to one or more binding sites such as binding site 210.

Referring back to FIG. 1, at process sequence 110 and FIG. 2C methods of the present disclosure include capturing one or more biomarkers-of-interest 220 on a substrate 200 configured to bind a plurality of biomarkers to a plurality of binding sites, wherein at least two binding sites such as binding site 210′ and 210″ are configured to bind at least two biomarkers, and wherein when one or more biomarkers are present forming one or more bound biomarkers-of-interest 226. In embodiments, a sample including one or more biomarkers-of-interest may be collected from a specimen, such as blood, blood serum, or dried blood. The sample may be contacted to substrate 200 under conditions in which one or more biomarkers-of-interest are able to contact and bind with one or more binding sites 210 as described here. In embodiments, the sample may be combined in a suitable buffer and flowed over the one or more binding sites.

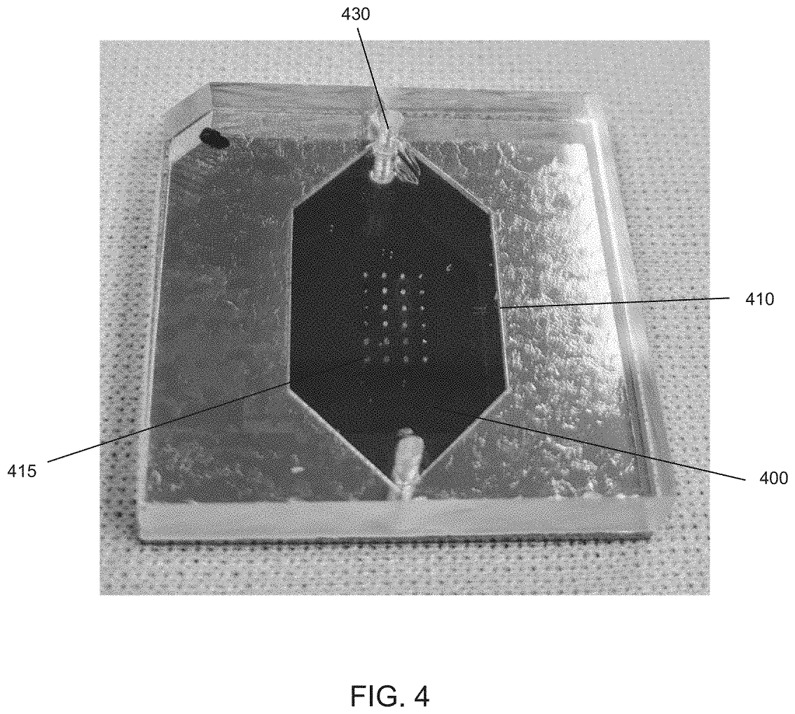

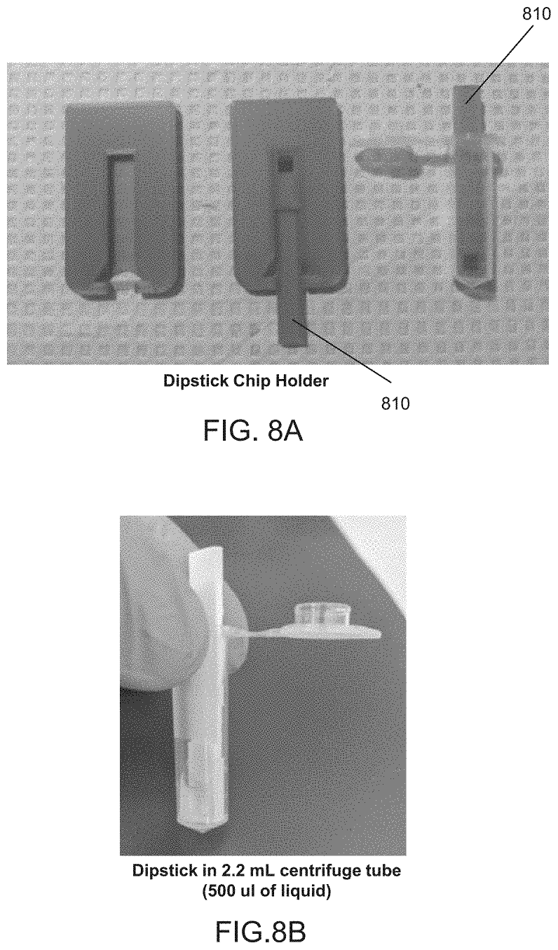

In some embodiments, the substrate 200 may be disposed within a cell such as a microfluidic flow cell, e.g., as shown in the photograph of FIG. 4 or a test-tube, e.g., as shown in FIG. 8B. In embodiments, a microfluidic flow cell such as GC-FP biochip 400 or test-tube is configured to accommodate a liquid such as a buffer solution. In embodiments, suitable buffer solution includes phosphate buffered saline supplemented with TWEEN-20 detergent (PBS-T). Referring to FIG. 4, a photograph of a GC-FP biochip 400 is shown including a gasket 410 and a window 415 forming a microfluidic chamber, where serum samples and other reagents can be applied through an opening 430 and flowed over the second layer 204 (not shown in FIG. 4). In embodiments, a biomarkers-of-interest, when present, will be in fluid communication with the one or more binding sites 210. Upon contact, a bond may form such that the biomarker-of-interest is specifically fixed upon or captured by a corresponding binding partner such as in the antigen-antibody reaction described above.

Referring to FIG. 1, at process sequence 120 and FIG. 2D, methods of the present disclosure include: contacting the one or more bound biomarkers-of-interest 226 with one or more fluorescent binding partners 240 to form one or more fluorescent complexes 245. In embodiments, one or more fluorescent binding partners 240 are provided under conditions sufficient to bind to one or more bound biomarkers-of-interest 226. In embodiments, a GC-FP analysis is performed wherein a gold-coated biochip such as GC-FP biochip 400 is contacted with a fluorophore-labelled secondary antibody suitable for coupling with a surface plasmon field to emit enhanced fluorescent signal. Non-limiting examples of one or more fluorescent binding partners include fluorescent binding partners capable of emitting a fluorescent signal such as a dye including fluorescently labeled anti-human antibodies such as anti-human IgG-ALEXA FLUOR® 647, or other suitable dye such as those described in U.S. Patent Application No. 20080233660 (herein incorporated by reference). In embodiments, anti-human secondary antibodies suitable for use herein include affinity-purified antibodies with specificity for human immunoglobulins. In embodiments, a dye is preselected to be pH-insensitive over a wide molar range. In embodiments, the fluorescent material is in a labeling solution including buffer and a secondary antibody labeled with fluorescent material sufficient to provide a degree of labeling for each conjugate in the amount of 2-8 fluorophore molecules per antibody molecule. In embodiments, after the application of the labeling material, the top surface of the second layer 204 may be flushed or washed with buffer solution to remove any free, or non-bound fluorescent material.

Referring to FIG. 1, at process sequence 130 and FIG. 3 methods of the present disclosure include contacting the substrate 200 with a source of collimated, polarized light 310, wherein the source 340 of collimated polarized light 310 is configured to emit light at a predetermined wavelength appropriate for transferring energy to a plurality of surface plasmons and excite fluorescence of the one or more fluorescent complexes 226. For example, in embodiments, collimated, polarized light 310 is applied to the second layer 204 at a predetermined angle of incidence. In embodiments, the second layer 204 is configured as a diffraction grating. In embodiments, in the region irradiated with the light, the surface plasmon generated at second layer 204 couples with the fluorescence of the one or more fluorescent complexes 245 to emit enhanced fluorescent signal. In embodiments, when a fluorescent material is present in the region irradiated with the light, the fluorescence material is excited by the enhanced electric field formed by surface plasmon resonance, and fluorescence is emitted. In embodiments, the fluorescent signal is enhanced by at least 10×, 100 to 5000×, such as 500 to 4000×, or 500 to 2000×.

Referring to FIG. 1, at process sequence 140 and FIG. 3 methods of the present disclosure include detecting emission light 350 of the one or more fluorescent complexes 245. In embodiments, the emitted fluorescent signal of emission light 350 is collected by a light detector such as a photodiode, a CCD image sensor, and detection optics 375. In embodiments, fluorescent enhancement can be achieved by preselecting a combination of wavelength and angle of incidence of the light 310.

Referring now to FIG. 1, at process sequence 150 methods of the present disclosure include determining a type and/or quantity of the plurality of biomarkers. In embodiments, the positioning of observed fluorescence may correspond to a predetermined type of biomarker. Thus, the binding sites are prepositioned on the substrate correlating to one or more specific biomarkers-of-interest. In embodiments, the fluorescence intensity as each binding site may correspond to the amount of detected biomarker such as detected antibody. In some embodiments, the amount of fluorescence intensity is determined by adjusting or normalizing image intensity such as that a positive control spot has an intensity of 100 (arbitrary units); finding the average fluorescence intensity of binding spots such as antigen spots (n=3 spots per chip); finding the average fluorescence intensity and standard deviation of one or more negative control spots (such as 2-3 spots of human serum albumin, aka: HSA); calculating a ratio of the (average binding spot intensity) vs. (negative control average spot intensity+3 standard deviations). In some embodiments, such as where testing for biomarkers-of-interest relating to COVID-19 such as antibodies specific to the polypeptides of SEQ ID NO:1, SEQ ID NO:2, or SEQ ID NO: 3, the calculating process sequence is repeated for each binding spot or antigen. In some embodiments, if 2 out of 3 of the ratios calculated are >1, then score=positive for antibodies against SARS CoV-2. In embodiments, if <2 out of 3 of the ratios are >1=negative for antibodies. In some embodiments, the stringency of the assay may be increased by requiring that at least 2 out of 3 are >1, and at least 1 ratio is >2.

In some embodiments, the present disclosure relates to a method for a qualitative and quantitative analysis of a plurality of biomarkers contained in a sample. In some embodiments, the quantitative and qualitative analysis are simultaneous. In some embodiments, the methods include capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest. In some embodiments, the methods include contacting the one or more bound biomarkers-of-interest with one or more fluorescent binding partners to form one or more fluorescent complexes. In some embodiments, the methods include contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In some embodiments, the methods include detecting emission light of the one or more fluorescent complexes. In some embodiments, the methods include determining a type and/or quantity of the plurality of biomarkers. In some embodiments, the one or more biomarkers are immunoglobulins. In some embodiments, the immunoglobulins include one or more of immunoglobulin G (IgG), immunoglobulin M (IgM), immunoglobulin A (IgA), immunoglobulin E (IgE), or isotypes or combinations thereof. In some embodiments, the one or more biomarkers are serum antibody targets. In some embodiments, the one or more biomarkers are antigens comprising: BBA69, BBA70, BmpA, DbpA, DbpB, ErpL, OspC, OspD, P41, P58, VIsE, or combinations hereof. In some embodiments, the substrate is disposed within a cell. In some embodiments, the cell is a fluidic flow cell or a test-tube. In some embodiments, each binding site of the plurality of binding sites includes two or more predetermined antigens configured to bind to two or more different predetermined biomarkers-of-interest. In some embodiments, the plurality of biomarkers are derived from blood serum, blood plasma, whole blood, or dried blood. In some embodiments, the type is characterized as immunoglobulin G (IgG), immunoglobulin M (IgM), immunoglobulin A (IgA), or isotypes or combinations thereof. In some embodiments, the type is further characterized as virus specific to SARS-COV-2. In some embodiments, the plurality of biomarkers are antibodies specific to a SARS-COV-2 full-length spike protein (SEQ ID NO:1), SARS-COV-2 spike 1 protein (SEQ ID NO: 2, SARS-COV-2 nuclear envelope protein (N) (SEQ ID NO: 3). In some embodiments, contacting the substrate with a source of collimated, polarized light, further includes positioning the substrate within a grating-coupled fluorescent plasmonic (GC-FP) detection platform. In some embodiments, the substrate is further characterized as a GC-FP assay chip. In some embodiments, the source of collimated polarized light is configured to excite fluorescence of the one or more fluorescent complexes greater than 10×, 100×, greater than 500×, or greater than 1000×. In some embodiments, detecting excitation light of the one or more fluorescent complexes further includes forming a fluorescent image on an antigen array or map of antigens upon the substrate. In some embodiments, the substrate is disposed atop a dipstick configured to be deposited into a cell characterized as a tube. In some embodiments, the substrate is a 4×4 mm GC-FP chip.

Referring now to FIG. 3, device 300 may be a tool suitable to perform methods for processing a substrate in accordance with some embodiments of the present disclosure. In embodiments, systems that may be used to perform the methods disclosed herein may include, but are not limited to, any of the GC-FP detection platforms, described in U.S. Pat. No. 8,368,897 to Reilly et al. (herein entirely incorporated by reference). In some embodiments, an apparatus such as device 300 for qualitative and quantitative analysis of a plurality of biomarkers contained in a sample, includes: a substrate such as substrate 200 described above including a plurality of binding sites, wherein two or more of the plurality of binding sites include two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes. In some embodiments, device 300 is a grating-coupled fluorescent plasmonic (GC-FP) detection platform such as described in U.S. Pat. No. 8,368,897, wherein the platform is configured for contacting the substrate such as substrate 200 with a source of collimated, polarized light, wherein the source of collimated polarized light is configured to emit light at a predetermined wavelength appropriate for transferring energy to a plurality of surface plasmons and excite fluorescence of the one or more fluorescent complexes as described above.

In some embodiments, the substrate 200 is disposed within a cell. Referring now to FIGS. 8A and 8B, in some embodiments, the substrate is disposed atop a dipstick and configured to fit within the cell, and the cell is configured as a tube such as a test-tube. In some embodiments, the device 300 is a GC-FP detection platform including a detector such as such as a photodiode, a CCD image sensor, and detection optics 375 configured to detect emission light from one or more fluorescent complexes when one or more biomarkers-of-interest are contacted with a plurality of bonding sites. In some embodiments, substrate incudes substrate 200 including a plurality of binding sites, wherein each binding site of the plurality of binding cites includes two or more predetermined antigens configured to bind to two or more predetermined antibodies-of-interest. In some embodiments, the plurality of biomarkers are serum derived antibodies, antibodies from whole blood, or antibodies from dried blood.

Still referring to FIG. 3, to facilitate control of the device 300 as described above, a controller 360 may be provided as any form of general-purpose computer processor that can be used in an industrial setting for controlling various aspects of device 300. The memory, or computer-readable medium, 356 of a CPU 352 may be one or more of readily available memory such as random-access memory (RAM), read only memory (ROM), floppy disk, hard disk, or any other form of digital storage, local or remote. In some embodiments, support circuits 354 are coupled to a CPU 352 for supporting the processor in a conventional manner. In embodiments, these circuits may include one or more of cache, power supplies, clock circuits, input/output circuitry and subsystems, and the like. In some embodiments, the methods disclosed herein may generally be stored in the memory 356 as a software routine 358 that, when executed by the CPU 352, causes the device 300 to perform processes of the present disclosure. The software routine 358 may also be stored and/or executed by a second CPU (not shown) that is remotely located from the hardware being controlled by the CPU 352. Some or all of the method of the present disclosure may also be performed in hardware. As such, the disclosure may be implemented in software and executed using a computer system, in hardware as, e.g., an application specific integrated circuit or other type of hardware implementation, or as a combination of software and hardware. The software routine 358 may be executed after the substrate 200 is positioned in device 300. The software routine 358, when executed by the CPU 352, transforms the general-purpose computer into a specific purpose computer (controller 360) that controls the device 300 operation such that the methods disclosed herein are performed. In some embodiments, the present disclosure includes a non-transient computer readable medium having instructions stored thereon that, when executed, causes an apparatus for qualitative and quantitative analysis of a plurality of biomarkers contained in a sample to perform a method, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the computer readable media is non-transitory computer readable media.

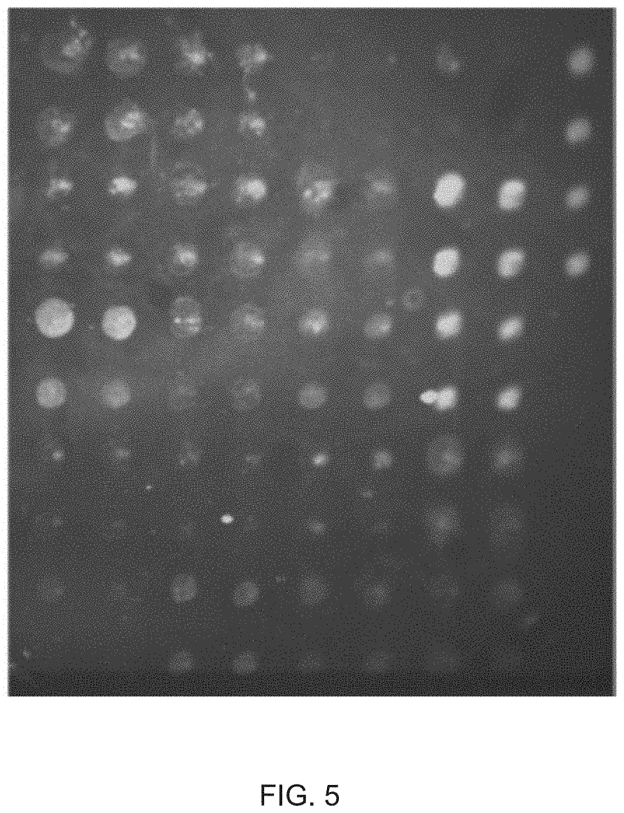

Referring now to FIG. 5, a photograph is shown of a GC-FP biochip image including a number of illuminated targets atop a plurality of binding sites. In embodiments, the fluorescence intensity at each binding site corresponds to the amount of detected antibody of various biomarkers-of-interest.

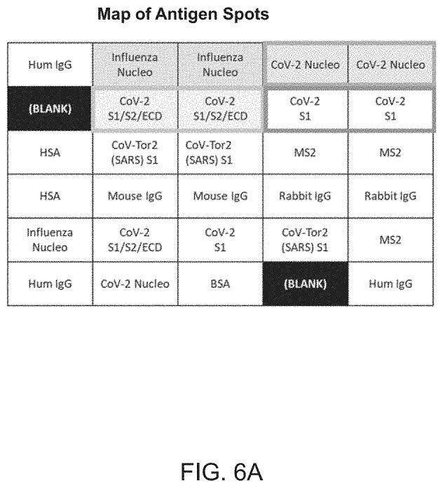

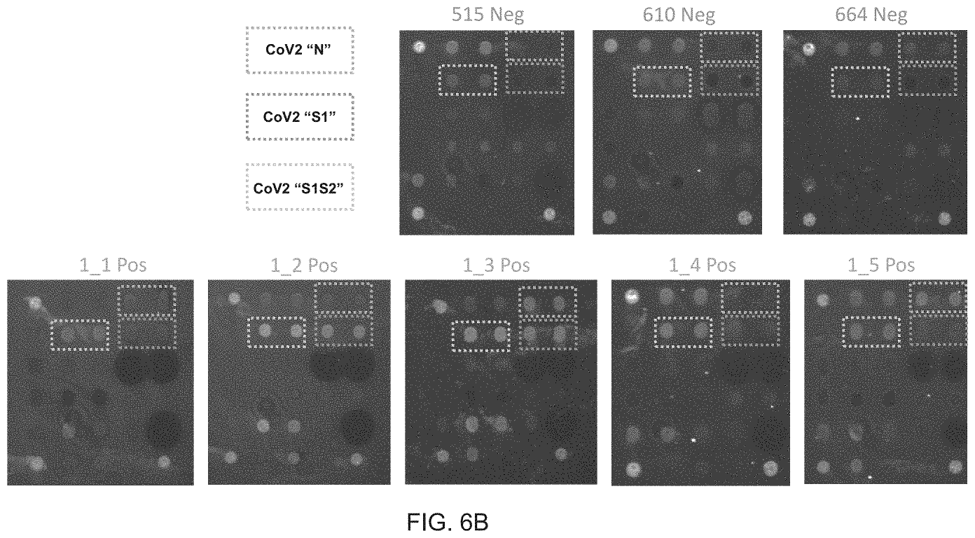

Referring now to FIGS. 6A and 6B, GC-FP chips processed with human blood serum are shown. FIG. 6A shows a map of antigen spots, and FIG. 6B shows images processed according to methods of the present disclosure. Three negative control samples (healthy individuals, with blood collected prior to COVID-19 pandemics were spotted onto binding sites at positions 515, 610, and 664. Five SARS-COV-2 positive samples were spotted (SARS-CoV-2 positive individuals confirmed with PCR test, all subjects were greater than 2 weeks convalescent). GC-FP enhanced fluorescent imaging of each chip for each sample was tested.

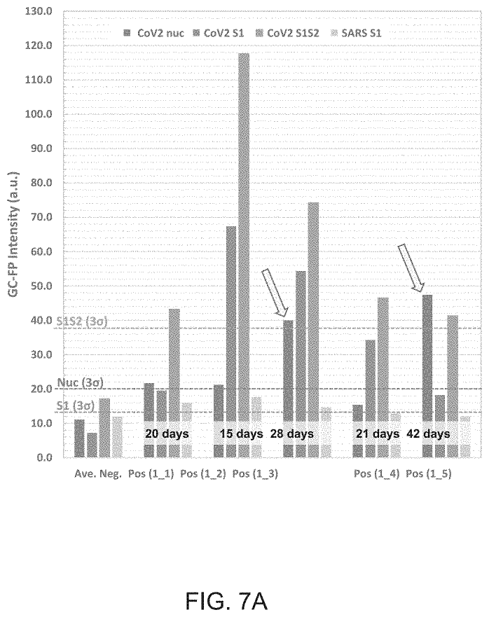

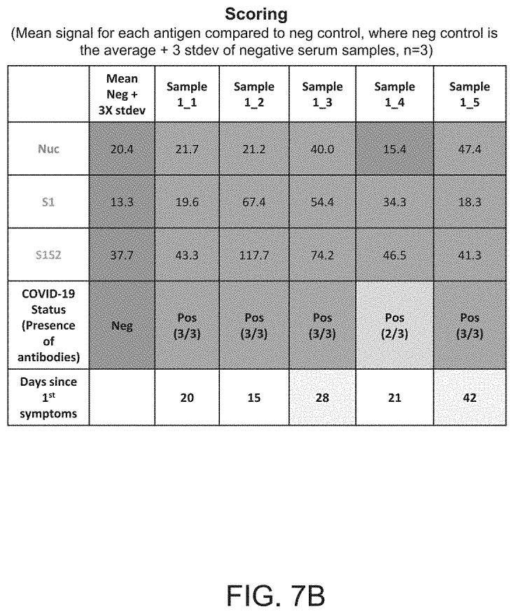

Referring now to FIGS. 7A and 7B, data from the images of FIGS. 6A and 6B was analyzed and scored. In embodiments, 3-sigma (Mean+3X std. deviation) for all three negative controls was determined for each target antigen (N, S1, and S1S2 of SARS-CoV-2). All positive samples were scored against these 3-sigma criteria. All positive samples showed greater than 3-sigma response for at least ⅔ of the target antigens (4 of 5 samples scored greater than 3-sigma for all 3 target antigens.

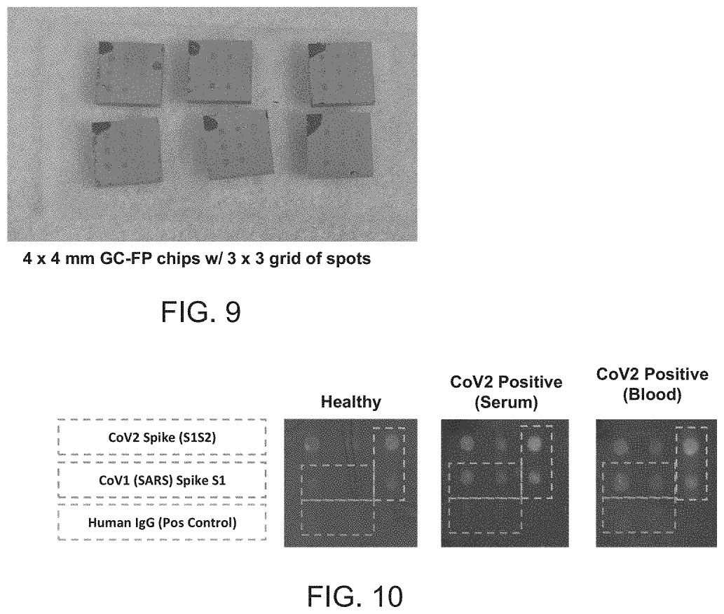

Referring now to FIGS. 8A and 8B a substrate 200 is shown disposed atop a dipstick 810. In embodiments, the dipstick is sized to fit within a 2.2 mL tube including at least 500 uL of liquid such as a buffer solution. Referring to FIG. 9 suitable substrate 200 are shown with predetermined binding spots suitable for antibody analysis in response to COVID-19. In embodiments, a 4×4 mm GC-FP chips are provided with 3×3 predetermined grid of binding sites or spots. In embodiments, the binding sites include one or more antigens for binding antibodies produced in a subject in response to SARS-CoV-2 infection. Referring to FIG. 10, images formed in accordance with the present disclosure are shown indicating negative or healthy, SARS-CoV-2 positive (from serum), and SARS-CoV-2 Positive from whole blood.

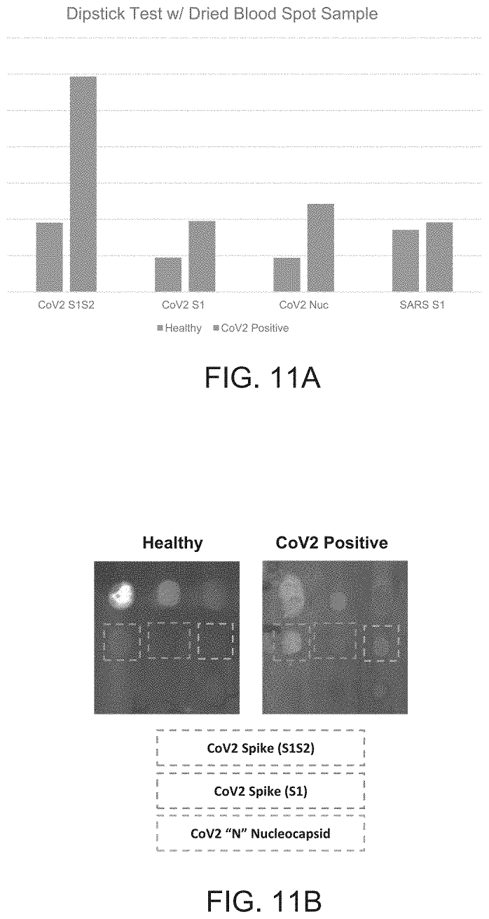

Referring now to FIG. 11, the dipstick format was applied to serum extracted from dried blood spots. In embodiments, dried blood spots were prepared on filter paper such as Whatman #4 paper (2 layers), Whatman 903 and stored longer than 24 hours at room temp or greater than 4 degrees Celsius. In embodiments, serum was extracted from 6 mm diameter dried blood spot samples (12 hrs. at 4 degrees Celsius) in a PBS-T buffer solution. In embodiments, the samples were applied to SARS-CoV-2 antibody test performed with dipstick GC-FP approach. Referring to FIGS. 11A and 11B, serum extracted from dried blood spots was a sufficient source for analyzing COVID-19 disease status. In embodiments, dried blood samples may be analyzed using a microfluidic chamber such as shown in FIG. 4.

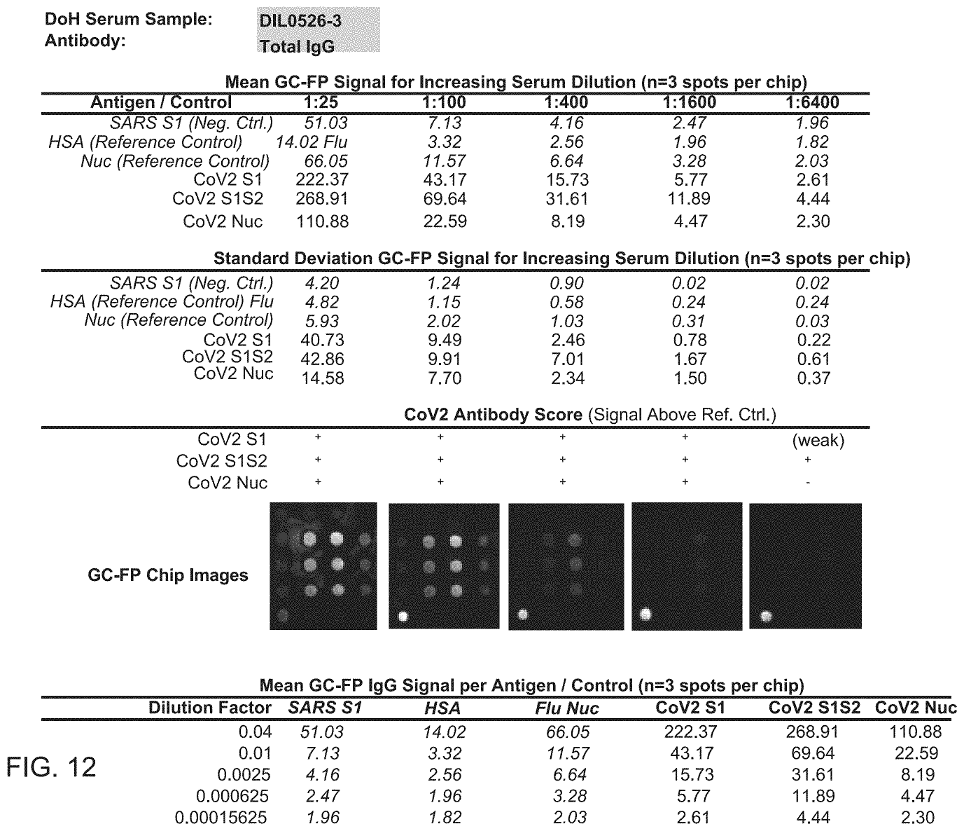

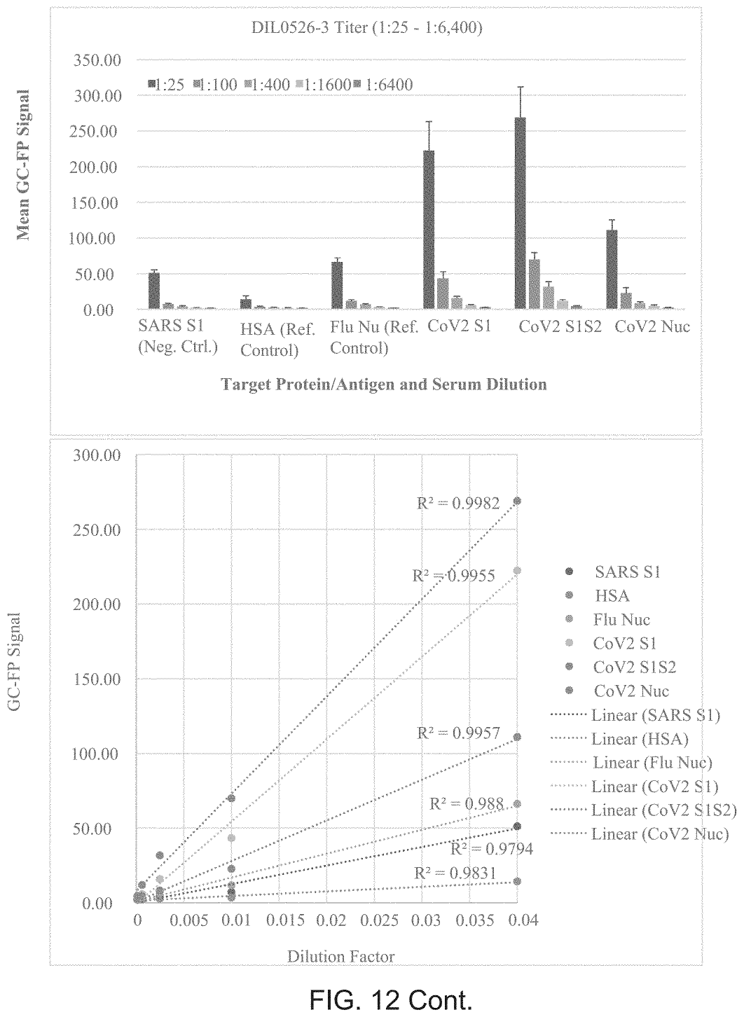

Referring to FIG. 12, GC-FP chips in accordance with the present disclosure were formed and provided robust indication of SARS-CoV-2.

In embodiments, the present disclosure relates to a method for a simultaneous qualitative and quantitative analysis of a plurality of biomarkers contained in a sample, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the one or more biomarkers are immunoglobulins. In embodiments, the immunoglobulins comprise one or more of immunoglobulin G (IgG), immunoglobulin M (IgM), immunoglobulin A (IgA), or isotypes or combinations thereof. In embodiments, the one or more biomarkers are serum antibody targets. In embodiments, the one or more biomarkers are antigens comprising: BBA69, BBA70, BmpA, DbpA, DbpB, ErpL, OspC, OspD, P41, P58, VIsE, or combinations hereof. In embodiments, the substrate is disposed within a cell. In embodiments, the cell is a fluidic flow cell or a test-tube. In embodiments, each binding site of the plurality of binding sites comprises two or more predetermined antigens configured to bind to two or more different predetermined biomarkers-of-interest. In embodiments, the plurality of biomarkers are derived from blood serum, blood plasma, whole blood, dried blood. In embodiments, the type is characterized as immunoglobulin G (IgG), immunoglobulin M (IgM), immunoglobulin A (IgA), or isotypes or combinations thereof. In embodiments, the type is further characterized as virus specific to SARS-COV-2. In embodiments, the plurality of biomarkers are antibodies specific to a SARS-COV-2 full-length spike protein (SEQ ID NO:1), SARS-COV-2 spike 1 protein (SEQ ID NO: 2, SARS-COV-2 envelope protein (N) (SEQ ID NO: 3). In embodiments, the substrate with a source of collimated, polarized light, further comprises positioning the substrate within a grating-coupled fluorescent plasmonic (GC-FP) detection platform. In embodiments, the substrate is further characterized as a GC-FP assay chip. In embodiments, the source of collimated polarized light is configured to cause fluorescent emission intensity greater than 10×, 100×, greater than 500×, or greater than 1000×. In embodiments, detecting emission light of the one or more fluorescent complexes further comprises forming a fluorescent image on an antigen array or map of antigens upon the substrate. In embodiments, the substrate is disposed atop a dip-stick configured to be deposited into a cell characterized as a tube. In embodiments, the substrate is a 4×4 mm GC-FP chip.

In embodiments, the present disclosure relates to an apparatus for qualitative and quantitative analysis of a plurality of biomarkers contained in a sample, including: a substrate comprising a plurality of binding sites, wherein two or more of the plurality of binding sites comprises two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes; and a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes. In embodiments, the substrate is disposed within a cell. In embodiments, the substrate is disposed atop a dip-stick and configured to fit within the cell, and wherein the cell is a test-tube. In embodiments, the GC-FP detection platform comprises a detector configured to detect emission light from one or more fluorescent complexes when one or more biomarkers-of-interest are contacted with a plurality of bonding sites. In embodiments, the substrate comprises a plurality of binding sites, wherein each binding site of the plurality of binding sites comprises two or more predetermined antigens configured to bind to two or more predetermined antibodies-of-interest. In embodiments, the plurality of biomarkers are serum derived antibodies, antibodies from whole blood, or antibodies from dried blood.

In embodiments, the present disclosure relates to a non-transitory computer readable medium having instructions stored thereon that, when executed, causes an apparatus for qualitative and quantitative analysis of a plurality of biomarkers contained in a sample to perform a method, including: capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers.

In embodiments, the present disclosure relates to methods for detecting and/or quantifying biomarkers in a specimen, such as from a blood, blood serum, or dried blood. In embodiments, the present disclosure relates to a method for a simultaneous qualitative and quantitative analysis of a plurality of biomarkers contained in a sample, including: capturing one or more biomarkers on a substrate to bind a plurality of biomarkers to a plurality of binding sites, wherein one or more binding sites bind at least one or more biomarkers, and wherein when one or more biomarkers are present, form one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light emits light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In some embodiments, the biomarkers are indicative of the present of disease, such as COVID-19, or the presence of SARS-CoV-2 or variants or fragments thereof.

In some embodiments, the present disclosure relates to an apparatus for qualitative and quantitative analysis of a plurality of biomarkers contained in a sample, including: a substrate including a plurality of binding sites, wherein one or more of the plurality of binding sites include one or more predetermined antigens that bind to one or more predetermined biomarkers-of-interest and form one or more bound biomarkers-of-interest when biomarkers-of-interest are flowed over the substrate, and wherein the biomarkers-of-interest, when present, bind to one or more fluorescent binding partners to form one or more fluorescent complexes; and a grating-coupled fluorescent plasmonic (GC-FP) detection platform, wherein the platform contacts the substrate with a source of collimated, polarized light, wherein the source of collimated polarized light emits light at a predetermined wavelength appropriate for transferring energy to a plurality of surface plasmons and excite fluorescence of the one or more fluorescent complexes.

In some embodiments, the present disclosure relates to a computer readable medium (such as non-transient) having instructions stored thereon that, when executed, causes an apparatus for qualitative and quantitative analysis of a plurality of biomarkers contained in a sample to perform a method, including: capturing one or more biomarkers on a substrate to bind a plurality of biomarkers to a plurality of binding sites, wherein at least one or more binding sites bind one or more biomarkers, and wherein when one or more biomarkers are present forming one or more bound biomarkers-of-interest; contacting the one or more bound biomarkers-of-interest with one or more fluorescent binding partners to form one or more fluorescent complexes; contacting the substrate with a source of collimated, polarized light, wherein the source of collimated polarized light is emits light at a predetermined wavelength appropriate for transferring energy to a plurality of surface plasmons and excite fluorescence of the one or more fluorescent complexes; detecting emission light of the one or more fluorescent complexes; and determining a type and/or quantity of the plurality of biomarkers. In embodiments, the computer readable media is non-transitory computer readable media.

### Example I

### Summary of Example I

The 2019 SARS CoV-2 (COVID-19) pandemic has illustrated the need for rapid and accurate diagnostic tests. Here, a multiplexed grating-coupled fluorescent plasmonics (GC-FP) biosensor platform was used to rapidly and accurately measure antibodies against COVID-19 in human blood serum and dried blood spot samples. The GC-FP platform measures antibody-antigen binding interactions for multiple targets in a single sample, and has 100% selectivity and sensitivity (n=23) when measuring serum IgG levels against three COVID-19 antigens (spike 51, spike S1S2, and the nucleocapsid protein). The GC-FP platform yielded a quantitative, linear response for serum samples diluted to as low as 1:1,600 dilution. Test results were highly correlated with two commercial COVID-19 antibody tests, including an enzyme linked immunosorbent assay (ELISA) and a Luminex-based microsphere immunoassay. To demonstrate test efficacy with other sample matrices, dried blood spot samples (n=63) were obtained and evaluated with GC-FP, yielding 100% selectivity and 86.7% sensitivity for diagnosing prior COVID-19 infection. The test was also evaluated for detection of multiple immunoglobulin isotypes, with successful detection of IgM, IgG and IgA antibody-antigen interactions. Last, a machine learning approach was developed to accurately score patient samples for prior COVID-19 infection, using antibody binding data for all three COVID-19 antigens used in the test.

### Introduction to Example I

The rapid spread of the 2019 SARS CoV-2 (COVID-19) virus has established an urgent need for accurate diagnostic technologies (See e.g., Pascarella, G., Strumia, A., Piliego, C., et al., 2020. Journal of Internal Medicine 288(2), 192-206). Due to the wide range in severity of this disease, many individuals remain asymptomatic or have mild symptoms, defining a population that is not tested at the time of acute infection. For these patients, the immune response to past COVID-19 infection is the best measure of exposure. Immune response to COVID-19 infection is variable, and may be linked to disease symptom severity, length of infection, and multiple patient-specific factors (See e.g., Sethuraman, N., Jeremiah, S. S., Ryo, A., 2020. JAMA 323(22), 2249-2251 and To, K. K.-W., Tsang, O. T.-Y., Leung, W.-S., et al., 2020. The Lancet Infectious Diseases 20(5), 565-574). Thus, quantitative detection of the antibody response to COVID-19 is critical to our response to this pandemic.

To measure antibody response to COVID-19 infection, a number of tests have been developed. Most tests detect binding of immunoglobulin G (IgG) and/or immunoglobulin M (IgM) to viral antigens. These tests are typically performed using whole blood, blood serum, or blood plasma. The most widely used approach is the enzyme linked immunosorbent assay (ELISA) (See e.g., Amanat, F., Stadlbauer, D., Strohmeier, S., et al., 2020. Nature Medicine 26(7), 1033-1036; Karp, D. G., Danh, K., Seftel, D., et al., 2020. medRxiv, 2020.2005.2029.20116004; and Randad, P. R., Pisanic, N., Kruczynski, K., et al., 2020. medRxiv, 2020.2005.2024.20112300). ELISA-based testing enables high throughput (processing many samples in parallel), but is typically limited to a single antigen per well (See e.g., Infantino, M., Damiani, A., Gobbi, F. L., et al., 2020. Isr Med Assoc J 22(4), 203-210; and Younes, N., Al-Sadeq, D. W., Al-Jighefee, H., et al., 2020. Viruses 12(6), 582).

Alternatively, multiplexed testing enables detection of immunoglobulin binding to more than one antigen within a single tube, well, plate or slide. Multiplexed tests include, but are not limited to microsphere immunoassays (MIAs) (Ayouba, A., Thaurignac, G., Morquin, D., et al., 2020. Journal of Clinical Virology 129, 104521). (See e.g, Randad, P. R., Pisanic, N., Kruczynski, K., et al., 2020. medRxiv, 2020.2005.2024.20112300), fluorescent protein microarrays (See Hedde, P. N., Abram, T. J., Jain, A., et al., 2020. Lab on a Chip 20(18), 3302-3309), and direct/label-free array technologies. (See Steiner, D. J., Cognetti, J. S., Luta, E. P., et al., 2020. bioRxiv, 2020.2006.2015.153064). Sample collection is a key challenge with implementing immunological/serological testing. Blood samples are typically obtained by venipuncture, followed by blood plasma or serum preparation. Alternatively, a simple finger stick and dried blood spotting allows self-collection, minimizing effort and likely increasing participation (See e.g., Au-Grüner, N., Au-Stambouli, O., Au-Ross, R. S., 2015. JoVE(97), e52619; Malsagova, K., Kopylov, A., Stepanov, A., et al., 2020. Diagnostics (Basel) 10(4), 248; Thevis, M., Knoop, A., Schaefer, M. S., et al., 2020. Drug Test Anal 12(7), 994-997; and Vazquez-Moron, S., Ardizone Jimenez, B., Jimenez-Sousa, M. A., et al., 2019. Scientific Reports 9(1), 7316). Samples collected in this manner may be maintained at ambient temperature and can be shipped using mail or courier service without the need for refrigeration. Dried blood spots (DBS) have been successfully utilized for immunological/serological testing for multiple viral diseases, including hepatitis C, HIV, and COVID-19 (See e.g., Karp, D. G., Danh, K., Seftel, D., et al., 2020. medRxiv, 2020.2005.2029.20116004; Malsagova, K., Kopylov, A., Stepanov, A., et al., 2020. Diagnostics (Basel) 10(4), 248; and Vazquez-Moron, S., Ardizone Jimenez, B., Jimenez-Sousa, M. A., et al., 2019. Scientific Reports 9(1), 7316). An enhanced fluorescence biosensor for multiplexed detection of antibodies for Lyme disease diagnosis has been described (See e.g., Chou, E., Lasek-Nesselquist, E., Taubner, B., et al., 2020. PLoS One 15(2), e0228772; Chou, E., Pilar, A., Guignon, E., et al., 2019. SPIE BiOS 10895; and Chou, E., Zenteno, G., Taubner, B., et al., 2018. SPIE Defense+Security 10629). Gold-coated nanoscale grating surfaces were modified with target antigens in a microarray format and then used to detect IgG or IgM binding from blood serum or plasma. Surface plasmons generated during illumination of the gold-coated biosensor chip actively enhance fluorescence emission intensity, yielding a high-sensitivity fluorescence detection platform. When measuring the fluorescence intensity of individual spots, the limit of detection of this approach was shown to be <2 ng/spot. This approach is referred to as “grating-coupled fluorescent plasmonics” (GC-FP). To achieve high sensitivity and an added measure of selectivity, fluorophore-tagged antibodies (against IgM or IgG) are applied during a labeling step. The entire detection process can be completed in less than 30 minutes with high sensitivity and specificity.

In embodiments, the GC-FP biosensor platform was used to develop a rapid immunoassay for simultaneous detection of antibodies against three COVID-19 spike protein antigens (receptor binding domain, RBD; spike 51 fragment; spike S1S2 extracellular domain) and the COVID-19 nucleocapsid protein (Nuc). Using serum, a 100% specificity and sensitivity was achieved for diagnosing prior COVID-19 infection, and a 100% specificity and sensitivity as high as 86.9% for DBS was demonstrated. For serum samples, GC-FP results are highly correlated with established testing methods (ELISA and MIA). The assay also has a large linear dynamic range across multiple orders of concentration. Because antibody titer against COVID-19 antigens is positively correlated with viral neutralization capacity (To, K. K.-W., Tsang, O. T.-Y., Leung, W.-S., et al., 2020. The Lancet Infectious Diseases 20(5), 565-574), the test may reveal the level of a subject's immune response.

### Materials and Methods

### Materials

Nucleocapsid protein (Nuc), the S1 fragment of the spike protein (S1), the extracellular domain of the spike protein (S1S2), the receptor binding domain of the spike protein (RBD), human serum albumin (HSA), the S1 domain of the 2005 SARS coronavirus spike protein (WH20 isolate, abbreviated “SARS-S1”), and human Influenza B nucleoprotein (B/Florida/4/2006 isolate “Flu Nuc”) were all obtained from Sino Biological, Inc. Positive control protein, human IgG protein (Hum IgG), SuperBlock blocking buffer and phosphate buffered saline (PBS) were obtained from ThermoFisher Scientific. PBS-TWEEN (PBS-T) solution consisting of PBS+0.05% v/v TWEEN-20 (Sigma-Aldrich) was prepared on a daily basis for all experiments. Alexa Fluor 647 labeled anti-human IgG (heavy and light chain) and anti-human IgM (heavy chain) were obtained from Invitrogen/ThermoFisher Scientific. Alexa Fluor 647 labeled anti-human IgA was obtained from Southern Biotech. ELISA testing was performed using COVID-19 human IgG testing kits from RayBiotech.

### Grating-Coupled Fluorescent Plasmonic (GC-FP) Biosensor Chip Preparation

In embodiments, GC-FP microchips were processed using the same conditions described previously, see e.g., Cady, N.C., Tokranova, N., Minor, A., et al., Multiplexed detection and quantification of human antibody response to COVID-19 infection using a plasmon enhanced biosensor platform, Biosens Bioelectron, 171, 112679 (2021) (herein entirely incorporated by reference). Gold coated grating-coupled fluorescent plasmonic (GC-FP) biosensor chips were fabricated as described in Chou, E., Lasek-Nesselquist, E., Taubner, B., et al., 2020. PLoS One 15(2), e0228772, and Chou, E., Pilar, A., Guignon, E., et al., 2019. SPIE BiOS 10895. GC-FP chips were printed with an array of 400 μm diameter spots of target and control antigens/proteins using an Arraylt SpotBot II microarray printer. Proteins/antigens were first diluted to 500 μg/μl in phosphate buffered saline (PBS) and then further diluted 1:1 just prior to printing with GBL protein array printing buffer (Grace Bio-Labs). For printing, a 180 μm diameter printing tip was used, at a relative humidity of 60-70%, at ambient temperature (˜25° C.). After printing, chips were allowed to dry at ambient temperature (˜25° C.) for 30 min, and were then transferred to a sealed container with desiccant for long-term storage (up to 4 weeks) before use.

### Biological Samples

Human blood samples were obtained from donors within New York state or from the Wadsworth Center, New York State Department of Health. Negative samples were collected prior to the 2019 SARS CoV-2 pandemic, and were obtained from the Lyme Disease Biobank. Additional blood samples were collected by finger stick. Lancet devices (27 ga.) and Whatman 903 protein saver collection cards were sent to volunteers with instructions. Blood sampling and testing was approved by the SUNY Polytechnic Institute Institutional Review Board (protocol #IRB-2020-10). Blood droplets were collected, allowed to dry, and then either hand delivered or mailed (via US Postal Service) to SUNY Polytechnic Institute. Following receipt of DBS samples, a sterile 6 mm diameter biopsy punch was used to remove samples from the collection cards. These disks were then soaked in 500 μl of PBS-T solution ˜12 hr at 4° C. with gentle rocking.

### GC-FP Detection Assay

Prior to performing GC-FP detection assays, GC-FP chips were filled with SuperBlock blocking buffer, then incubated at room temperature for 15 min. Chips were then placed in a custom fluidic apparatus to provide sequential flow of sample and reagents using the following steps: 1) 500 μl of PBS-T at 100 μl/min, 2) 400 μl of diluted human blood serum or extracted dried blood spot sample at 50 μl/min, 3) 500 μl of PBS-T at 100 μl/min, 4) 400 μl of Alexa 647 anti-human IgG/IgM (diluted 1:400 in PBS-T) at 100 μl/min, and 5) 500 μl of PBS-T at 100 μl/min. GC-FP chips were then analyzed in a customized Ciencia, Inc. fluorescent plasmonic imaging instrument. For serum testing, a standard dilution of serum in PBS-T (1:25) was used. For dried blood spot testing, undiluted extract from the 6 mm diameter segment of the blood collection card was used in place of serum. Ciencia image analysis LabView software was used to define a region of interest (ROI) for each individual spot on the GC-FP biosensor chip and the fluorescence intensity of each spot was measured. The fluorescence intensity of all spots was normalized to the human IgG (Hum IgG) internal control spots on each chip, to account for variability between individual chips and individual experiments.

### Data Analysis

Normalized spot intensity data was exported from the software and further analyzed using GraphPad Prism 8.0 software (for fitting, ROC analysis, correlation, and statistical analysis). To account for variation between chips and experiments, normalized intensity data for positive control and COVID-19 antigen spots (mean intensity, x̅) were divided by the average negative control spot intensity, plus three times the standard deviation (a) of the negative control spot intensity (x̅+3σ) to produce a detection metric as follows:

$$GC - FP Detection Ratio = \frac{\overline{x} target spot intensity}{( \overline{x} neg . ctrl . spot intensity ) + ( 3 σ neg . ctrl . spot intensity )} \qquad Equation 1$$

A support vector machine (SVM) based machine learning approach was used to analyze GC-FP detection data, and was implemented with freely available SVM software (LibSVM—http://www.csie.ntu.edu.tw/˜cjlin/libsvm) (See e.g., Chang, C.-C., Lin, C.-J., 2011. ACM Transactions on Intelligent Systems and Technology 2, Article 27). The nu-SVC package within LibSVM was utilized with sigmoid kernel, and a grid search for cost and gamma parameters was conducted to maximize the prediction accuracy of the SVM model.

### Results and Discussion

Detection Assay Development and Characterization

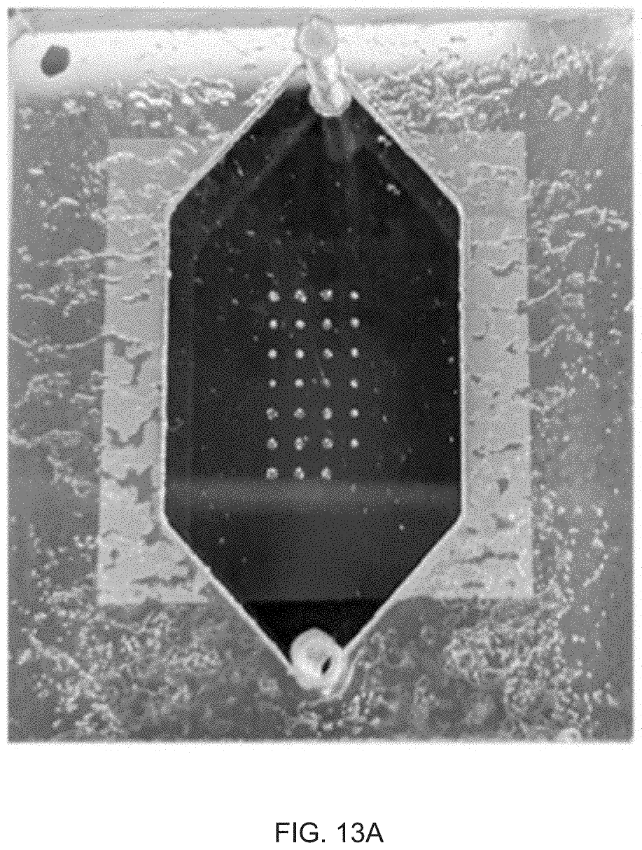

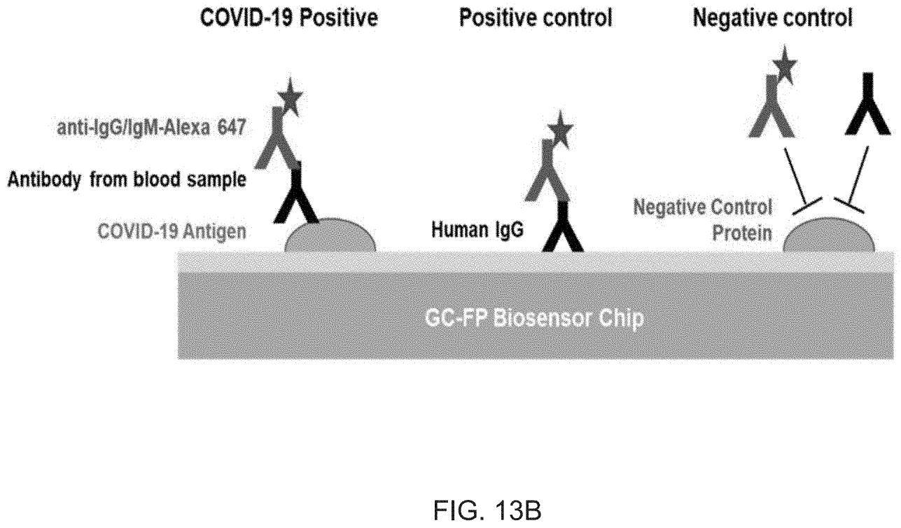

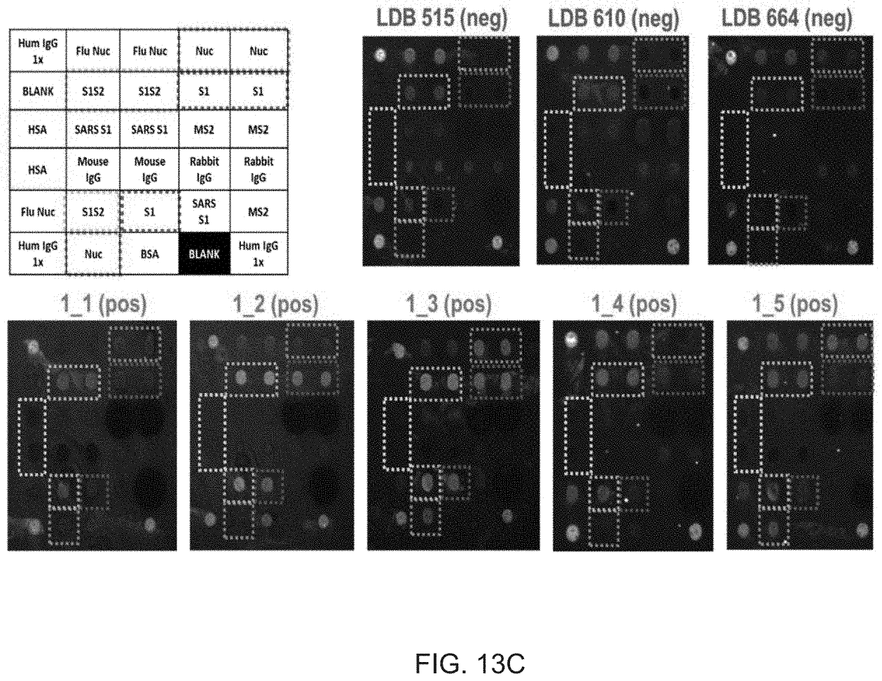

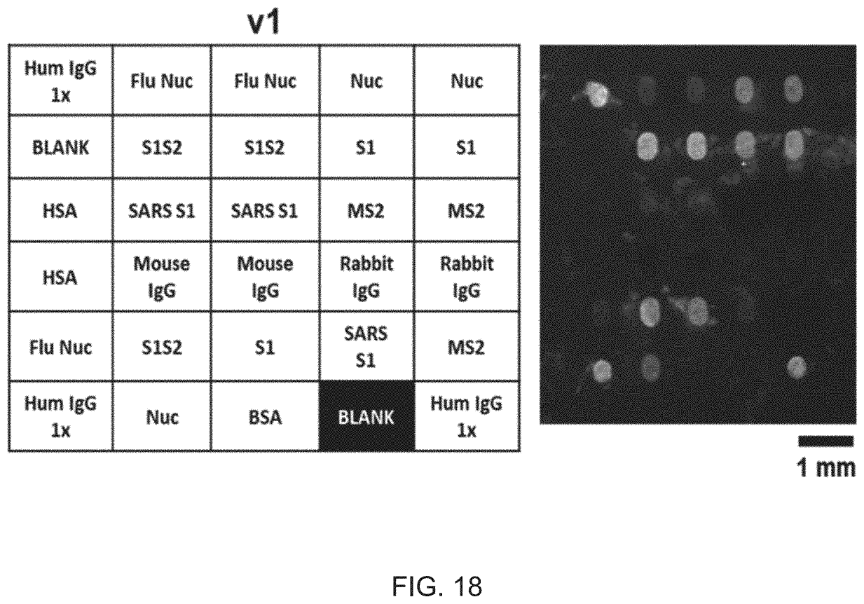

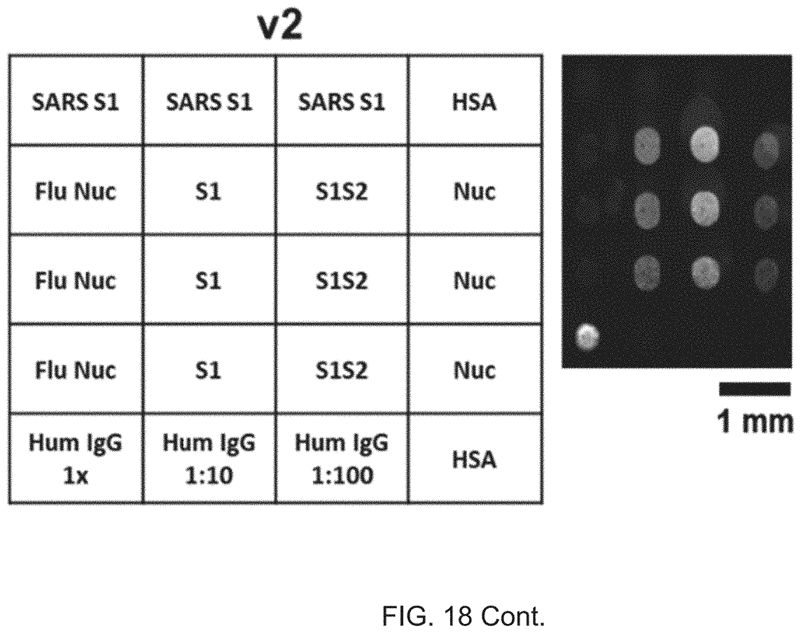

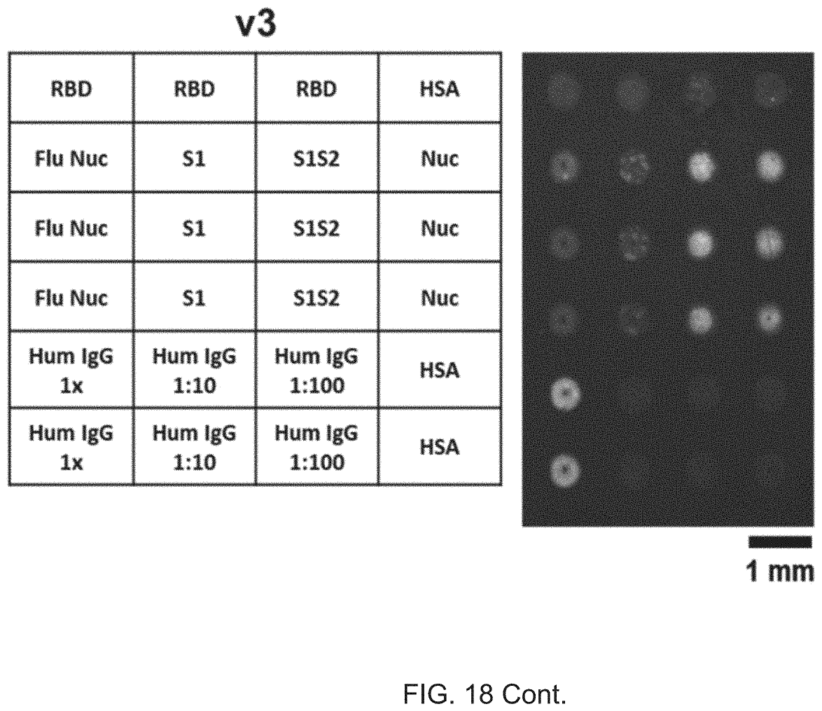

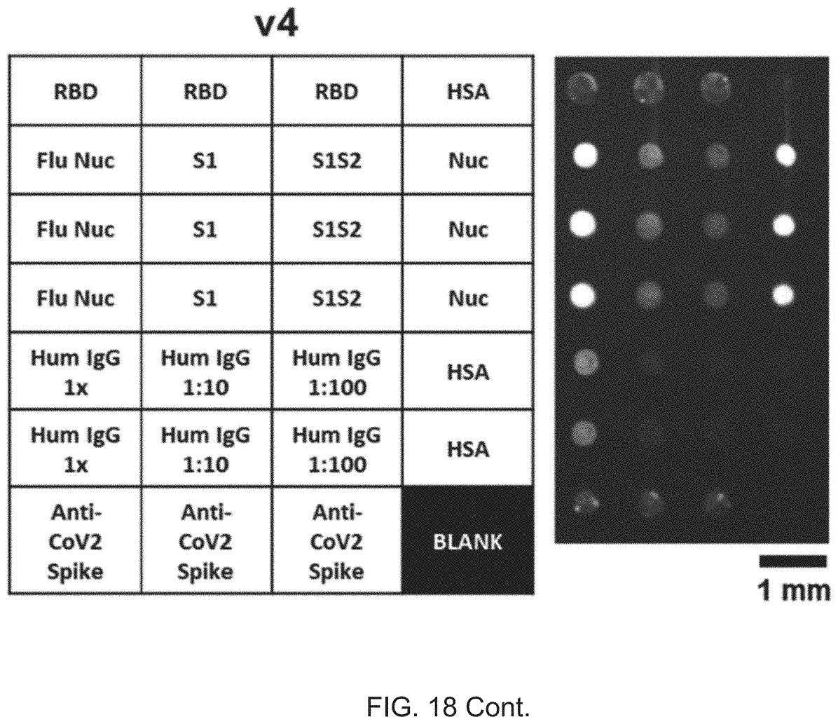

Rapid (less than 30 min), multiplexed detection of immunoglobulin binding to COVID-19 antigens was performed using our previously described GC-FP biosensing approach and a Ciencia, Inc. fluorescent plasmonic imaging instrument (Chou, E., Lasek-Nesselquist, E., Taubner, B., et al., 2020. PLoS One 15(2), e0228772; Chou, E., Pilar, A., Guignon, E., et al., 2019. SPIE BiOS 10895; and Chou, E., Zenteno, G., Taubner, B., et al., 2018. SPIE Defense+Security 10629). An example GC-FP microchip for COVID-19 immunological analysis is shown in FIG. 13A. COVID-19 specific antigens and control proteins were immobilized on the GC-FP biosensor chips in a variety of configurations (v1-v4, FIG. 18). Testing utilized serum samples from subjects previously infected with COVID-19 who were expected to have an antibody response to COVID-19 antigens. All subjects had fully recovered from infection and were more than 2 weeks convalescent. Negative control samples showed no GC-FP response for IgG binding to COVID-19 Nuc and S1 antigens, and in some cases, very weak response for full-length spike S1S2 extracellular domain antigen (FIG. 13C).

FIG. 18 depicts four different versions of the GC-FP biosensor chip were developed. The arrangement of protein/antigen spots is shown for each chip version, as well as a representative GC-FP fluorescence image.

More specifically, referring to FIGS. 13A-13C depict: a GC-FP biosensor chip shown with gasket and fluidic cover attached (FIG. 13A); COVID-19 antigens or control proteins were spotted onto GC-FP biosensor chips, then assessed for antibody binding from human blood samples. Subsequent labeling with Alexa Fluor 647 tagged anti-human IgG was used for the enhanced fluorescence detection output (FIG. 13B); enhanced fluorescence images of GC-FP biosensor chips (v1) processed with negative control serum (blood serum collected >2 years prior to the COVID-19 pandemic and serum from subjects who were >2 weeks convalescent from PCR-confirmed COVID-19 infection (FIG. 13C). Boxes with dotted outlines indicate paired spots of key COVID-19/SARS CoV-2 antigens, S1, S1S2, and Nuc.

### GC-FP Antibody Detection in Human Serum and Dried Blood Spot Samples

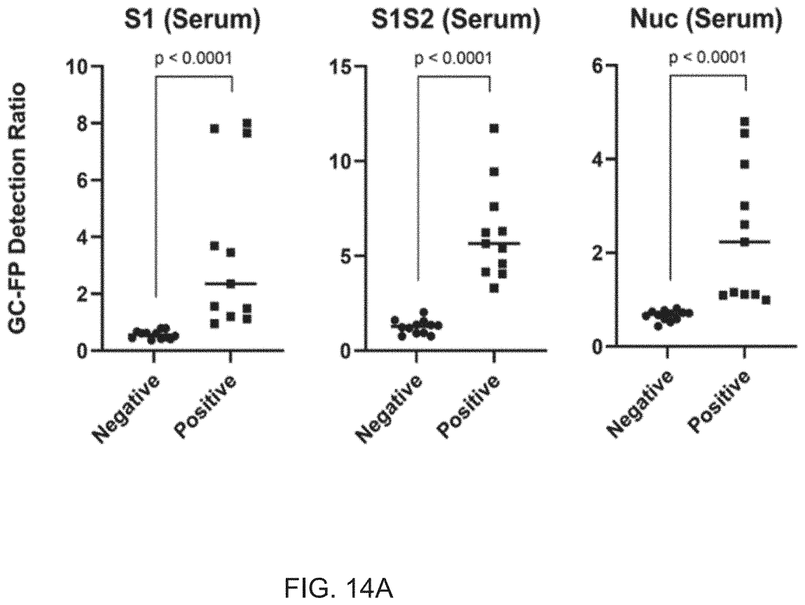

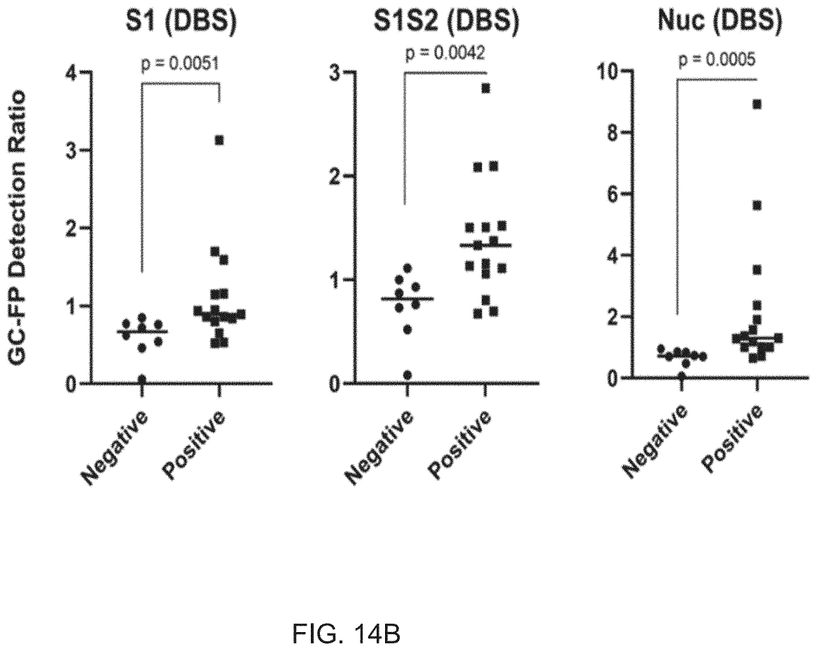

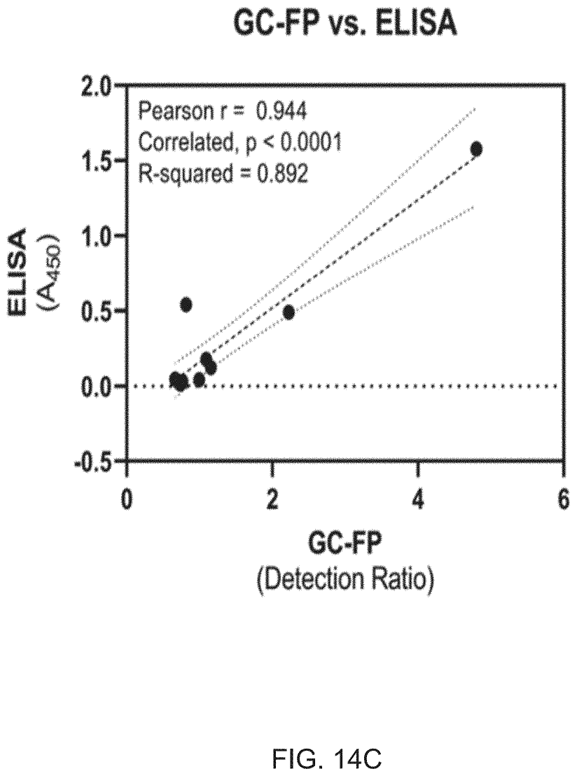

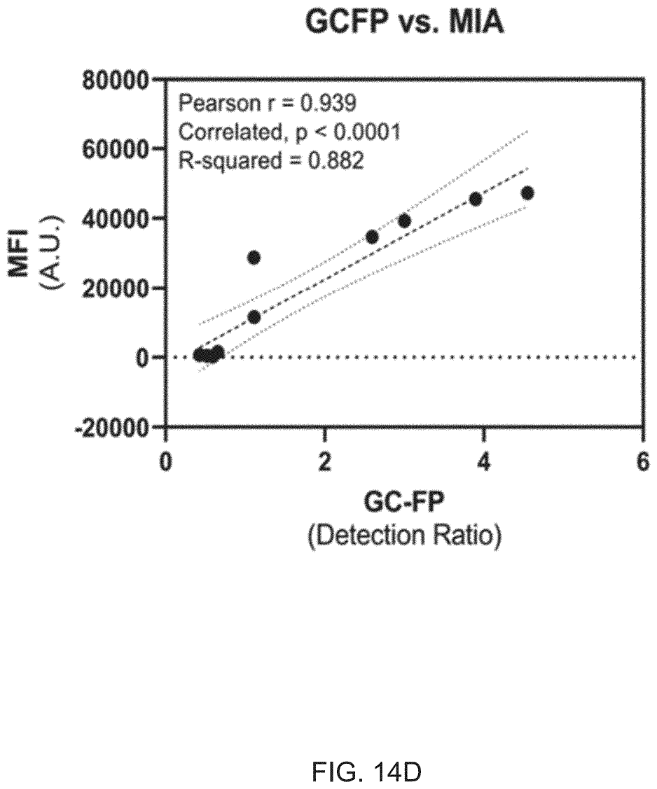

Using v2, v3 and v4 GC-FP chips, 23 different human blood serum samples, and 24 dried blood spot samples (with verified infection status) were tested. Samples were tested at a dilution rate of 1 part serum to 25 parts PBS-T (for serum) or undiluted (for dried blood spot extracts). Raw GC-FP fluorescence intensity data were normalized as described in the Materials and Methods section, and a “GC-FP detection ratio” was calculated to account for chip-to-chip differences and any variability in processing conditions. The GC-FP detection ratio provides a measure of signal above background, while also accounting for spot-to-spot variability. The results of these experiments are shown in FIG. 14, Table 1, and Table 2. The average GC-FP detection ratio for COVID-19 positive samples vs. COVID-19 negative samples was significant for all antigens, for both serum and dried blood spot samples (Mann-Whitney U-test, p<0.05).

Table I depicts GC-FP detection ratio data for serum and dried blood spot samples with verified infection status. ELISA and Luminex MIA data are shown for serum samples tested with these assays. Samples scored positive (GC-FP detection ratio above the ROC threshold) are denoted by bold text. The Nuc diagnostic score and machine learning (ML) score are shown for dried blood spot samples. GC-FP detection ratios for serum samples were compared to ELISA and a Luminex-based MIA (Yang et al. 2020). As shown in FIGS. 14C and 14D, the measured GC-FP detection ratios for serum samples were highly correlated with both ELISA absorbance values (Pearson r=0.944, R-squared=0.892) and MIA fluorescence intensity (MFI) values (Pearson r=0.939, R-squared=0.882). These results demonstrate that the GC-FP detection approach provides comparable immunodetection results to established, gold-standard methods.

TABEL 1
Serum Samples

| | | | | Nuc- | | |
|---|---|---|---|---|---|---|
| | | | | ELISA | Nuc-MIA | |
| Sample | | | | (score/ | (score/ | |
| ID | S1 | S1S2 | Nuc | A450) | MFI) | Notes |
| 2_1 | 0.67 | 1.55 | 0.81 | neg/0.054 | n/a | no known exposure |
| 2_2 | 0.61 | 0.94 | 0.72 | neg/0.027 | n/a | no known exposure |
| 2_3 | 0.79 | 0.77 | 0.74 | neg/0.013 | n/a | no known exposure |
| 2_4 | 0.79 | 0.77 | 0.67 | neg/0.046 | n/a | no known exposure |
| 2_S | 0.52 | 1.33 | 0.77 | neg/0.032 | n/a | no known exposure |
| 2_6 | 0.61 | 1.60 | 0.70 | neg/0.031 | n/a | no known exposure |
| 2_7 | 0.62 | 1.37 | 0.71 | neg/0.037 | n/a | no known exposure |
| DIL0526-26 | 0.42 | 0.92 | 0.59 | n/a | neg/259 | 2009 pre-COVID |
| DIL0526-27 | 0.41 | 1.20 | 0.52 | n/a | neg/421 | 2009 pre-COVID |
| DIL0526-28 | 0.45 | 1.23 | 0.65 | n/a | neg/1,563 | 2009 pre-COVID |
| DIL0526-29 | 0.48 | 2.03 | 0.59 | n/a | neg/238 | 2009 pre-COVID |
| DIL0526-30 | 0.37 | 1.36 | 0.43 | n/a | neg/719 | 2009 pre-COVID |
| 1_1 | 1.20 | 3.31 | 1.16 | pos/0.123 | n/a | Positive by RT-PCR |
| 1_2 | 3.69 | 5.65 | 1.09 | pos/0.178 | n/a | Positive by RT-PCR |
| 1_3 | 7.65 | 11.73 | 4.80 | pos/1.577 | n/a | Positive by RT-PCR |
| 1_4 | 1.49 | 5.41 | 0.99 | neg/0.043 | n/a | Positive by RT-PCR |
| 1_5 | 1.56 | 4.59 | 2.23 | pos/0.491 | n/a | Positive by RT-PCR |
| D1L0526-3 | 7.81 | 9.44 | 3.89 | n/a | pos/45,543 | Positive by RT-PCR |
| DIL0526-5 | 8.00 | 7.61 | 4.55 | n/a | pos/47,301 | Positive by RT-PCR |
| DIL0526-8 | 3.45 | 6.23 | 3.00 | n/a | pos/39,255 | Positive by RT-PCR |
| DIL0526-13 | 2.35 | 4.06 | 1.11 | n/a | pos/11,577 | Positive by RT-PCR |
| DIL0526-18 | 1.12 | 6.31 | 2.60 | n/a | pos/34,715 | Positive by RT-PCR |
| DIL0526-21 | 0.95 | 4.16 | 1.11 | n/a | pos/28,806 | Positive by RT-PCR |

Dried Blood Spot Samples (Verified Infection Status)

| Sample | | | | (score/ | (score/ | |
|---|---|---|---|---|---|---|
| ID | S1 | S1S2 | Nuc | A450) | MFI) | Notes |
| COV_5 | 0.54 | 0.73 | 0.70 | neg | neg | Negative by RT-PCR |
| COV_6 | 0.72 | 1.11 | 0.70 | neg | neg | Negative by RT-PCR |
| COV_7 | 0.06 | 0.08 | 0.06 | neg | neg | Negative by RT-PCR |
| COV_9 | 0.85 | 1.00 | 0.95 | neg | neg | Negative by RT-PCR |
| COV_14 | 0.46 | 0.52 | 0.48 | neg | neg | Negative by RT-PCR |
| COV_16 | 0.77 | 0.93 | 0.84 | neg | neg | Negative by RT-PCR |
| COV_18 | 0.62 | 0.76 | 0.74 | neg | neg | Negative by RT-PCR |
| COV_12 | 0.76 | 0.87 | 0.85 | neg | neg | IgG and IgM negative |
| COV_2 | 0.65 | 0.80 | 0.72 | neg | neg | Positive by RT-PCR |
| COV_3 | 1.59 | 1.13 | 5.63 | pos | pos | Positive by RT-PCR |
| COV_13 | 0.53 | 0.67 | 0.65 | neg | neg | Positive by RT-PCR |
| COV_17 | 0.94 | 1.11 | 1.01 | pos | pos | Positive by RT-PCR |
| COV_28 | 0.80 | 1.37 | 3.53 | pos | pos | Positive by RT-PCR |
| COV_29 | 1.70 | 1.51 | 1.57 | pos | pos | Positive by RT-PCR |
| COV_40 | 1.15 | 1.50 | 2.37 | pos | pos | Positive by RT-PCR |
| COV_41 | 0.86 | 1.06 | 1.02 | pos | pos | Positive by RT-PCR |
| COV_43 | 0.52 | 0.70 | 1.19 | pos | neg | Positive by RT-PCR |
| COV_44 | 3.13 | 2.85 | 8.92 | pos | pos | Positive by RT-PCR |
| NYC_1 | 1.16 | 2.09 | 1.29 | pos | pos | Positive by RT-PCR |
| NYC_5 | 0.95 | 2.09 | 1.90 | pos | pos | Positive by RT-PCR |
| COV_19 | 0.89 | 1.52 | 1.37 | pos | pos | IgG pos., no PCR test |
| COV_38 | 0.86 | 1.33 | 1.30 | pos | pos | IgG pos., no PCR test |
| COV_24 | 0.84 | 1.16 | 1.01 | pos | pos | IgM pos., no PCR test |

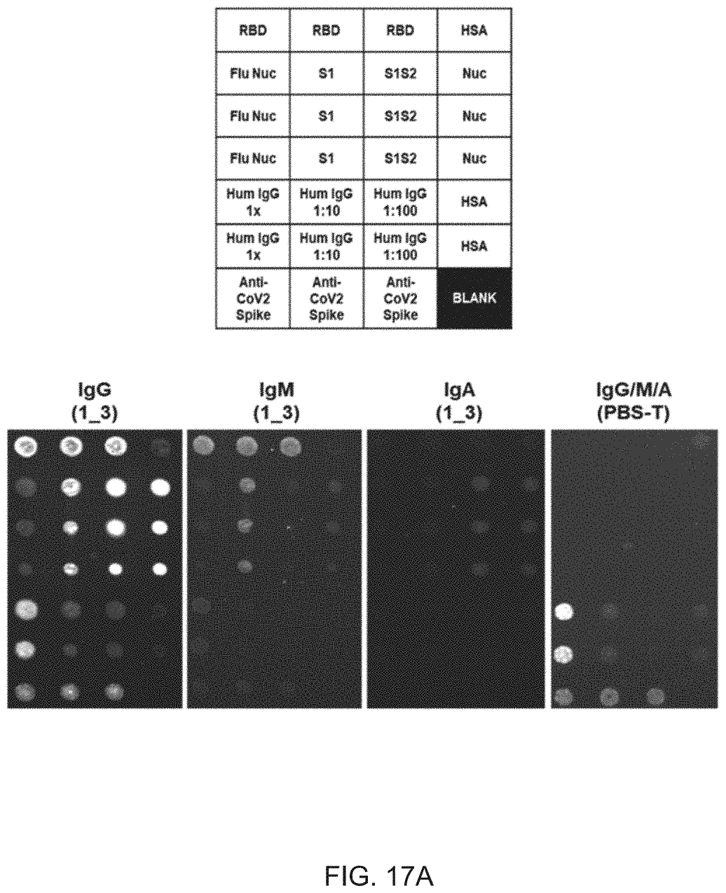

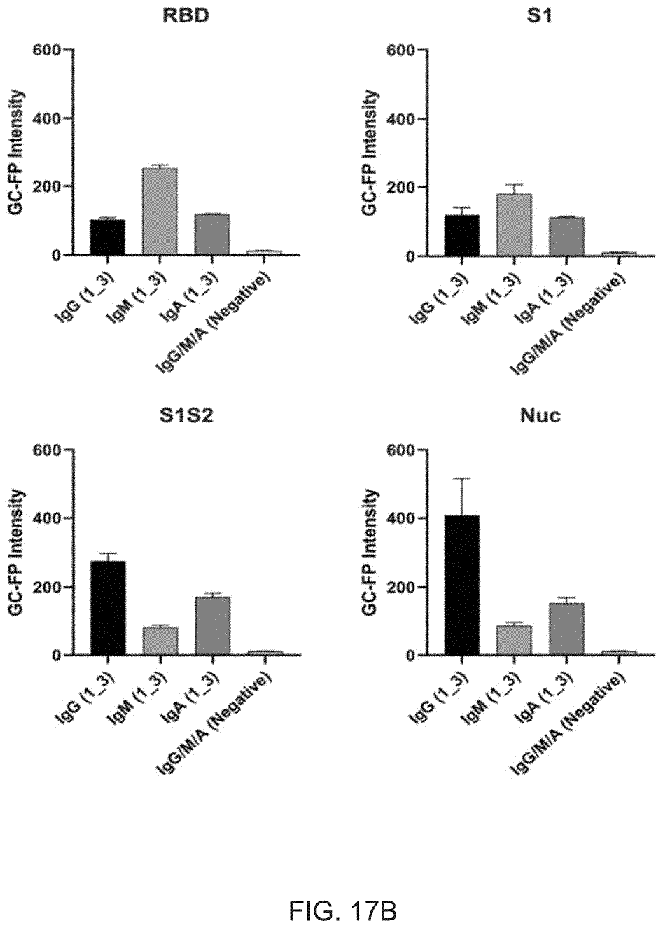

Referring to FIGS. 17A and 17B, serum sample 1_3 was tested on three separate GC-FP biosensor chips that included RBD, S1, S1S2, and Nuc antigens. A fourth chip was processed using PBS-T as a negative control. The three chips tested with 1_3 serum were labeled with Alexa 647-tagged anti-human IgG, anti-human IgM, or anti-human IgA, while the negative control chip was labeled with a mixture of all three secondary antibodies. Mean GC-FP intensity (n=3 spots per chip) is shown, for each COVID-19 antigen.

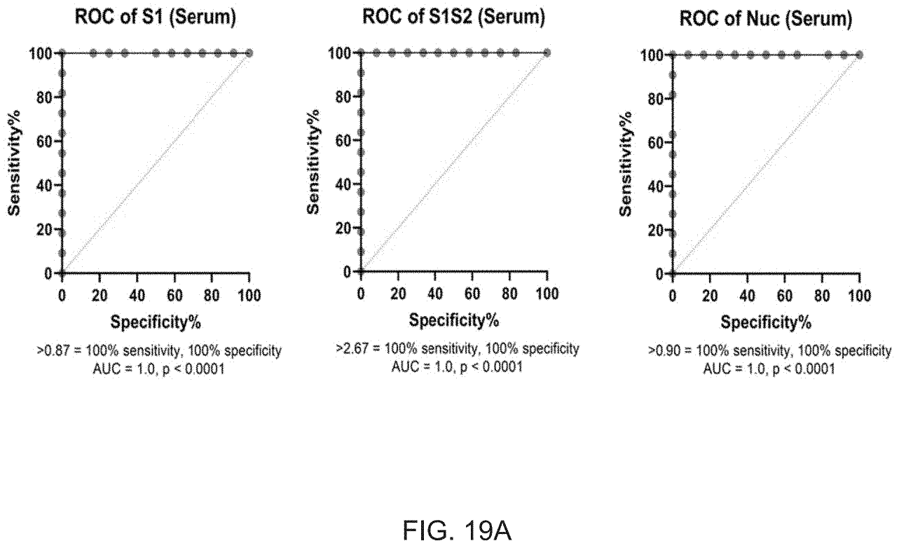

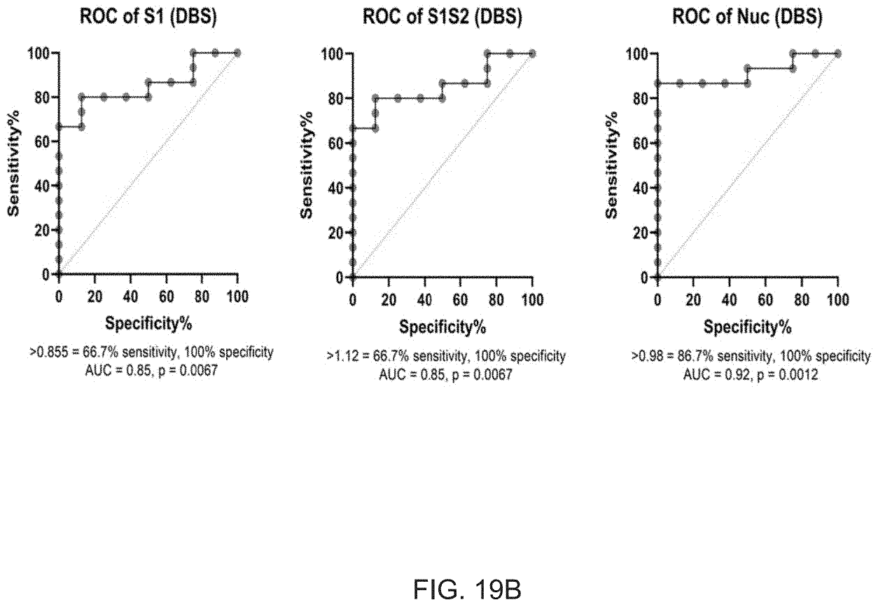

To determine if individual IgG/antigen responses could be used for diagnostic purposes, receiver operator characteristic (ROC) analysis was performed (FIGS. 19A and 19B). For serum samples, 100% sensitivity and specificity could be achieved when the following GC-FP detection ratio thresholds were met: S1=0.87, S1S2=2.67, Nuc=0.9. For dried blood spot samples, ROC analysis yielded 100% specificity and variable sensitivity when the following GC-FP detection ratio thresholds were met: S1=0.855, S1S2=1.12, and Nuc=0.98. When these GC-FP detection ratio thresholds were exceeded (to maintain 100% specificity) assay sensitivity was relatively low for S1 and S1S2 antigens (66.7% for both) but increased to 86.7% for the nucleocapsid antigen (Nuc).

More specifically FIGS. 19A and 19B depict receiver operator characteristic (ROC) analysis for serum (FIG. 19A) and dried blood spot (FIG. 19B) samples with known COVID-19 infection history. The area under the curve (AUC) was calculated for each plot and the associated p value for this analysis is shown. The GC-FP detection ratio needed for 100% specificity and maximum sensitivity is also reported for each plot.

The observed reduction in sensitivity for DBS vs. serum samples could be due to the sample format, especially since sample stability and extraction are more variable for dried blood spots vs. blood serum. Other reasons for the reduction in sensitivity could be variability in individual immune responses and/or reporting consistency for the research subjects who provided samples. Other studies have shown that individual antibody responses are variable (See e.g, Amanat, F., Stadlbauer, D., Strohmeier, S., et al., 2020. Nature Medicine 26(7), 1033-1036), which would also affect assay sensitivity. Importantly, no false positives were observed within the limited number of samples that were tested.

In addition to testing samples with known COVID-19 infection history, 39 additional dried blood spot samples were received and tested. For these samples, information was provided about exposure to infected individuals, potential disease symptoms, or complete lack of exposure, but none of the subjects had been tested with a COVID-19 RT-PCR test. The results from GC-FP testing for these samples are shown in TABLE 2. Due to the fact that COVID-19 infection status was unverified for these samples, sensitivity and specificity was not determined.

TABLE 2
Serum samples (Verified COVID-19 status)

| | | | | Nuc- | Nuc- | |
|---|---|---|---|---|---|---|
| | | | | ELISA | MIA | |
| Sample | | | | (score/ | (score/ | |
| ID | S1 | S1S2 | Nuc | A450) | MFI) | Notes |
| 2_1 | 0.67 | 1.55 | 0.81 | neg/0.054 | n/a | presumed negative, |
| | | | | | | no known exposure |
| 2_2 | 0.61 | 0.94 | 0.72 | neg/0.027 | n/a | presumed negative, |
| | | | | | | no known exposure |
| 2_3 | 0.79 | 0.77 | 0.74 | neg/0.013 | n/a | presumed negative, |
| | | | | | | no known exposure |
| 2_4 | 0.79 | 0.77 | 0.67 | neg/0.046 | n/a | presumed negative, |
| | | | | | | no known exposure |
| 2_5 | 0.52 | 1.33 | 0.77 | neg/0.032 | n/a | presumed negative, |
| | | | | | | no known exposure |
| 2_6 | 0.61 | 1.60 | 0.70 | neg/0.031 | n/a | presumed negative, |
| | | | | | | no known exposure |
| 2_7 | 0.62 | 1.37 | 0.71 | neg/0.037 | n/a | presumed negative, |
| | | | | | | no known exposure |
| DIL0526-26 | 0.42 | 0.92 | 0.59 | n/a | neg/259 | 2009 pre-COVID |
| DIL0526-27 | 0.41 | 1.20 | 0.52 | n/a | neg/421 | 2009 pre-COVID |
| DIL0526-28 | 0.45 | 1.23 | 0.65 | n/a | neg/1,563 | 2009 pre-COVID |
| DIL0526-29 | 0.48 | 2.03 | 0.59 | n/a | neg/238 | 2009 pre-COVID |
| DIL0526-30 | 0.37 | 1.36 | 0.43 | n/a | neg/719 | 2009 pre-COVID |
| 1_1 | 1.20 | 3.31 | 1.16 | pos/0.123 | n/a | Positive by RT-PCR |
| 1_2 | 3.69 | 5.65 | 1.09 | pos/0.178 | n/a | Positive by RT-PCR |
| 1_3 | 7.65 | 11.73 | 4.80 | pos/1.577 | n/a | Positive by RT-PCR |
| 1_4 | 1.49 | 5.41 | 0.99 | neg/0.043 | n/a | Positive by RT-PCR |
| 1_5 | 1.56 | 4.59 | 2.23 | pos/0.491 | n/a | Positive by RT-PCR |
| DIL0526-3 | 7.81 | 9.44 | 3.89 | n/a | pos/45,543 | Positive by RT-PCR |
| DIL0526-5 | 8.00 | 7.61 | 4.55 | n/a | pos/47,301 | Positive by RT-PCR |
| DIL0526-8 | 3.45 | 6.23 | 3.00 | n/a | pos/39,255 | Positive by RT-PCR |
| DIL0526-13 | 2.35 | 4.06 | 1.11 | n/a | pos/11,577 | Positive by RT-PCR |
| DIL0526-18 | 1.12 | 6.31 | 2.60 | n/a | pos/34,715 | Positive by RT-PCR |
| DIL0526-21 | 0.95 | 4.16 | 1.11 | n/a | pos/28,806 | Positive by RT-PCR |
| ROC | 0.87 | 2.67 | 0.9 | | | |
| threshold | | | | | | |

Dried Blood Spots (Verified COVID-19 status)

| | | | | Nuc | | |
|---|---|---|---|---|---|---|
| Sample | | | | ROC | ML | |
| ID | S1 | S1S2 | Nuc | Score | Score | Notes |
| COV_5 | 0.54 | 0.73 | 0.70 | neg | neg | Negative by RT-PCR |
| COV_6 | 0.72 | 1.11 | 0.70 | neg | neg | Negative by RT-PCR |
| COV_7 | 0.06 | 0.08 | 0.06 | neg | neg | Negative by RT-PCR |
| COV_9 | 0.85 | 1.00 | 0.95 | neg | neg | Negative by RT-PCR |
| COV_14 | 0.46 | 0.52 | 0.48 | neg | neg | Negative by RT-PCR |
| COV_16 | 0.77 | 0.93 | 0.84 | neg | neg | Negative by RT-PCR |
| COV_18 | 0.62 | 0.76 | 0.74 | neg | neg | Negative by RT-PCR |
| COV_12 | 0.76 | 0.87 | 0.85 | neg | neg | IgG and IgM negative |
| COV_2 | 0.65 | 0.80 | 0.72 | neg | neg | Positive by RT-PCR |
| COV_3 | 1.59 | 1.13 | 5.63 | pos | pos | Positive by RT-PCR |
| COV_13 | 0.53 | 0.67 | 0.65 | neg | neg | Positive by RT-PCR |
| COV_17 | 0.94 | 1.11 | 1.01 | pos | pos | Positive by RT-PCR |
| COV_28 | 0.80 | 1.37 | 3.53 | pos | pos | Positive by RT-PCR |
| COV_29 | 1.70 | 1.51 | 1.57 | pos | pos | Positive by RT-PCR |
| COV_40 | 1.15 | 1.50 | 2.37 | pos | pos | Positive by RT-PCR |
| COV_41 | 0.86 | 1.06 | 1.02 | pos | pos | Positive by RT-PCR |
| COV_43 | 0.52 | 0.70 | 1.19 | pos | neg | Positive by RT-PCR |
| COV_44 | 3.13 | 2.85 | 8.92 | pos | pos | Positive by RT-PCR |
| NYC_1 | 1.16 | 2.09 | 1.29 | pos | pos | Positive by RT-PCR |
| NYC_5 | 0.95 | 2.09 | 1.90 | pos | pos | Positive by RT-PCR, |
| | | | | | | IgG indeterminate |
| COV_19 | 0.89 | 1.52 | 1.37 | pos | pos | IgG positive, |
| | | | | | | no PCR test |
| COV_38 | 0.86 | 1.33 | 1.30 | pos | pos | IgG positive, |
| | | | | | | no PCR test |
| COV_24 | 0.84 | 1.16 | 1.01 | pos | pos | IgG positive, |
| | | | | | | no PCR test |
| ROC | 0.855 | 1.12 | 0.98 | | | |
| threshold | | | | | | |

Dried Blood Spots (Unverified COVID-19 status)

| | | | | Nuc | | |
|---|---|---|---|---|---|---|
| Sample | | | | ROC | ML | |
| ID | S1 | S1S2 | Nuc | Score | Score | Notes |
| COV_1 | 0.43 | 1.67 | 0.70 | neg | pos | presumed negative, |
| | | | | | | no known exposure |
| COV_4 | 0.60 | 0.76 | 0.81 | neg | neg | presumed negative, |
| | | | | | | no known exposure |
| COV_8 | 0.76 | 0.90 | 0.86 | neg | neg | presumed negative, |
| | | | | | | no known exposure |
| COV_10 | 0.79 | 0.86 | 0.87 | neg | neg | presumed negative, |
| | | | | | | no known exposure |
| COV_11 | 0.88 | 0.99 | 0.96 | neg | neg | presumed negative, |
| | | | | | | no known exposure |
| COV_15 | 0.52 | 0.59 | 0.59 | neg | neg | presumed negative, |
| | | | | | | no known exposure |
| COV_20 | 0.82 | 0.91 | 0.92 | neg | neg | presumed negative, |
| | | | | | | no known exposure |
| COV_21 | 0.69 | 0.81 | 0.81 | neg | neg | presumed negative, |
| | | | | | | no known exposure |
| COV_22 | 0.69 | 0.82 | 0.81 | neg | neg | presumed negative, |
| | | | | | | no known exposure |
| COV_23 | 0.82 | 0.88 | 0.89 | neg | neg | presumed negative, |
| | | | | | | no known exposure |
| COV_25 | 0.60 | 0.69 | 0.63 | neg | neg | presumed negative, |
| | | | | | | no known exposure |
| COV_26 | 0.62 | 0.82 | 0.79 | neg | neg | presumed negative, |
| | | | | | | no known exposure |
| COV_27 | 0.48 | 0.75 | 0.57 | neg | neg | presumed negative, |
| | | | | | | no known exposure |
| COV_30 | 0.41 | 0.60 | 0.56 | neg | neg | presumed negative, |
| | | | | | | no known exposure |
| COV_31 | 0.32 | 0.64 | 0.47 | neg | neg | presumed negative, |
| | | | | | | no known exposure |
| COV_32 | 0.65 | 0.81 | 0.72 | neg | neg | presumed negative, |
| | | | | | | no known exposure |
| COV_33 | 0.46 | 0.64 | 0.54 | neg | neg | presumed negative, |
| | | | | | | no known exposure |
| COV_34 | 0.58 | 0.71 | 0.71 | neg | neg | presumed negative, |
| | | | | | | no known exposure |
| COV_35 | 0.43 | 0.58 | 0.53 | neg | neg | presumed negative, |
| | | | | | | no known exposure |
| COV_36 | 0.75 | 1.06 | 0.89 | neg | neg | presumed negative, |
| | | | | | | no known exposure |
| COV_37 | 0.78 | 1.40 | 0.99 | pos | pos | presumed negative, |
| | | | | | | no known exposure |
| COV_39 | 0.40 | 0.58 | 0.57 | neg | neg | presumed negative, |
| | | | | | | no known exposure |
| COV_42 | 0.53 | 0.72 | 0.68 | neg | neg | presumed negative, |
| | | | | | | no known exposure |
| NYC_9 | 0.73 | 0.92 | 1.43 | pos | pos | presumed negative, |
| | | | | | | no known exposure |
| NYC_12 | 0.58 | 0.68 | 1.30 | pos | neg | presumed negative, |
| | | | | | | no known exposure |
| VT_3 | 0.70 | 0.96 | 1.04 | pos | neg | presumed negative, |
| | | | | | | no known exposure |
| VT_4 | 0.75 | 0.91 | 1.04 | pos | neg | presumed negative, |
| | | | | | | no known exposure |
| NYC_2 | 0.53 | 0.82 | 1.80 | pos | pos | Lives with COVID |
| | | | | | | positive subject, |
| | | | | | | asymptomatic |
| NYC_3 | 1.43 | 2.03 | 1.46 | pos | pos | Lives with COVID |
| | | | | | | positive subject, |
| | | | | | | asymptomatic |
| NYC_6 | 0.73 | 0.94 | 1.15 | pos | neg | Lives with COVID |
| | | | | | | positive subject, |
| | | | | | | asymptomatic |
| NYC_4 | 1.22 | 2.43 | 2.37 | pos | pos | Lives with COVID |
| | | | | | | positive subject, |
| | | | | | | symptomatic |
| NYC_7 | 1.17 | 2.16 | 1.32 | pos | pos | Lives with COVID |
| | | | | | | positive subject, |
| | | | | | | symptomatic |
| NYC_8 | 2.55 | 3.09 | 2.30 | pos | pos | Lives with COVID |
| | | | | | | positive subject, |
| | | | | | | symptomatic |
| NYC_10 | 0.63 | 2.94 | 1.73 | pos | pos | International travel |
| | | | | | | during pandemic, |
| | | | | | | minor symptoms |
| NYC_11 | 0.65 | 1.27 | 2.22 | pos | pos | International travel |
| | | | | | | during pandemic, |
| | | | | | | minor symptoms |
| VT_1 | 0.90 | 1.87 | 1.97 | pos | pos | Lives with |
| | | | | | | symptomatic subject, |
| | | | | | | minor symptoms |
| VT_2 | 0.80 | 1.03 | 2.46 | pos | pos | Symptomatic, but no |
| | | | | | | COVID test |
| VT_5 | 0.75 | 1.07 | 1.37 | pos | pos | Exposed to subject |
| | | | | | | w/symptoms but |
| | | | | | | no COVID test |
| VT_6 | 0.70 | 1.31 | 1.48 | pos | pos | Exposed to subject |
| | | | | | | w/symptoms but |
| | | | | | | no COVID test |
| ROC | 0.855 | 1.12 | 0.98 | | | |
| threshold | | | | | | |

### Multiplexed Data Analysis

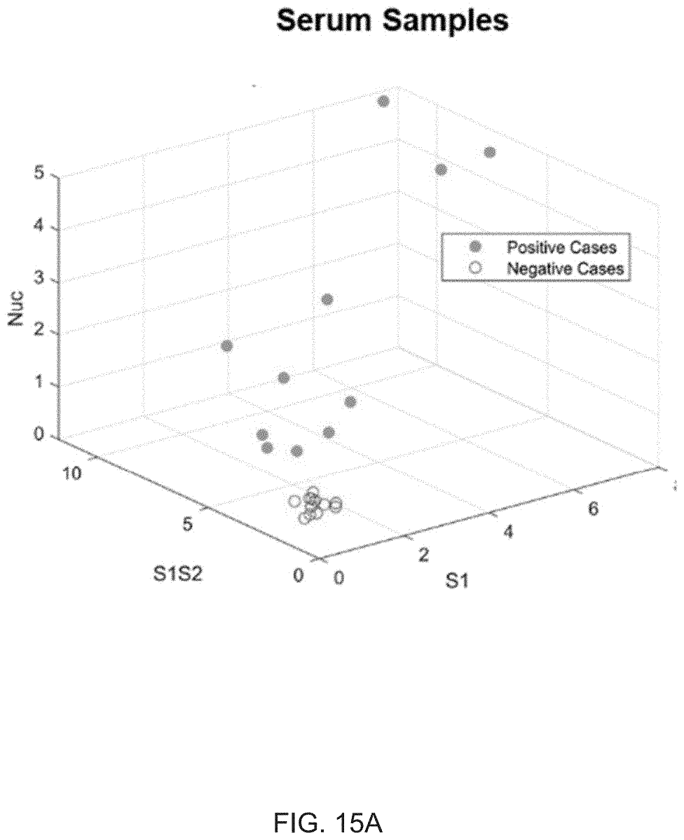

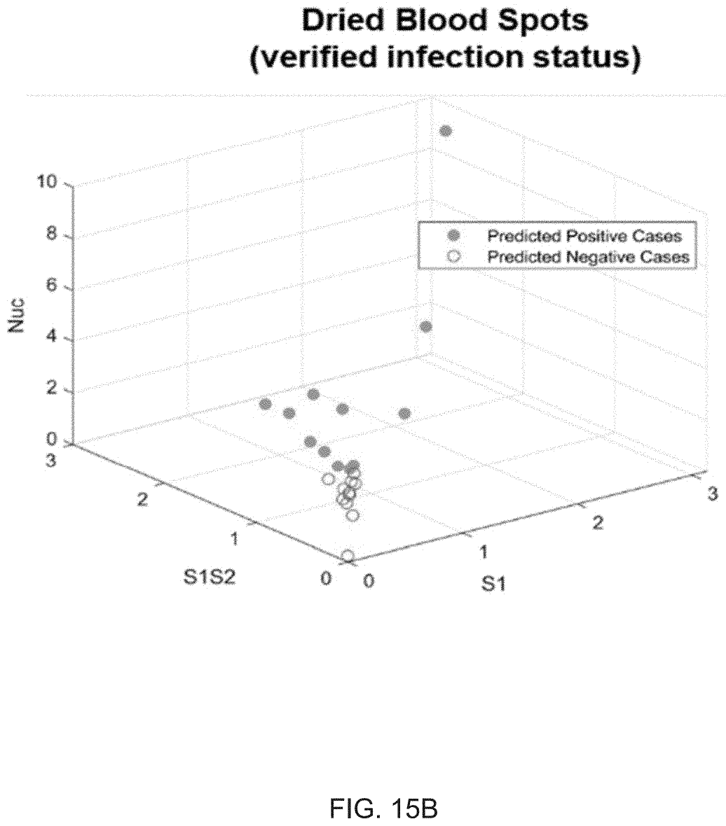

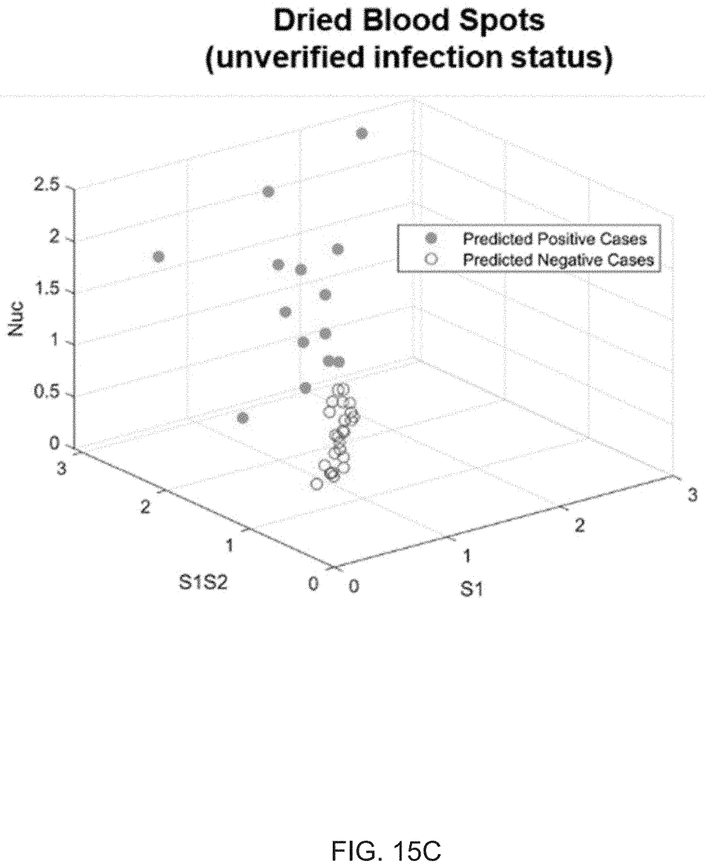

Scoring samples based on a composite antibody response (to all antigens) could provide increased diagnostic accuracy and a more complete understanding of a subject's antibody response to infection. To this end, a support vector machine (SVM) based machine learning (ML) approach was used to differentiate and classify samples based on their antibody response to three target antigens (S1, S1S2, and Nuc). ML approaches have been used extensively for classification and diagnosis when data from multiple biomarkers or targets is available (See e.g., Sarkar, D., Saha, S., 2019. Journal of Biosciences 44(4), 104; and Uddin, S., Khan, A., Hossain, M. E., et al., 2019. BMC Medical Informatics and Decision Making 19(1), 281). SVM software (LibSVM) (Chang, C.-C., Lin, C.-J., 2011. ACM Transactions on Intelligent Systems and Technology 2, Article 27) was trained using GC-FP data from serum samples (Table 1). After training, the ML model was challenged with 10-fold cross-validation on unlabeled serum data, yielding 100% selectivity and sensitivity, which matched ROC analysis for individual antigens. After training and validation, DBS data were classified with the SVM model (FIGS. 15B & 15C, Table 1, Table 2). For dried blood spots with verified prior infection status, the SVM model classified samples with 80% sensitivity and 100% selectivity. For dried blood spots with unverified prior infection status, SVM classification resulted in better correlation with presumed infection status than when scoring with individual antibody responses (S1, S1S2, or Nuc). While many additional samples will be needed to fully train the ML model, and to understand the true selectivity and sensitivity of the GC-FP assay, here the potential for ML based scoring antibody responses to multiple antigens has been demonstrated.

Referring to FIGS. 15A-15C, visualization of testing data following machine learning-based analysis is shown. The GC-FP detection ratio data for all serum samples were plotted as a function of S1, S1S2, and Nuc in FIG. 15A. An SVM ML model was used to classify dried blood spot sample data from (FIG. 15B) subjects with verified COVID-19 infection status, and (FIG. 15C) subjects with unverified COVID-19 infection status.

Quantification of Antibody Titer and Comparison to ELISA

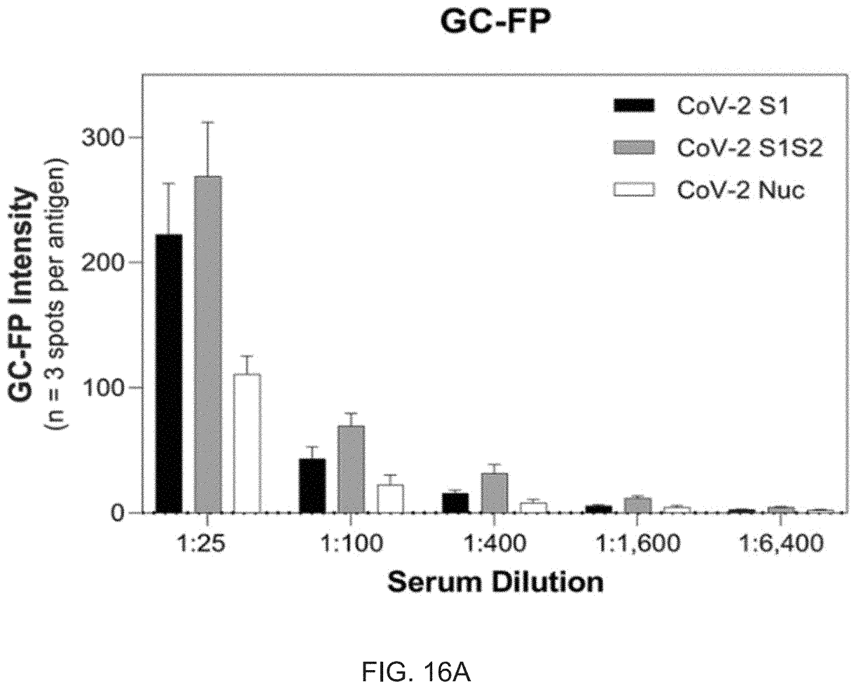

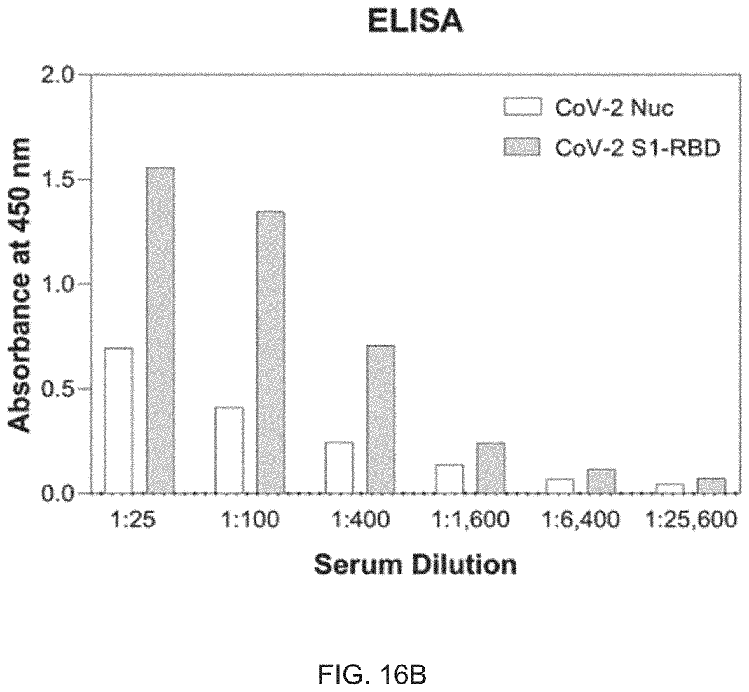

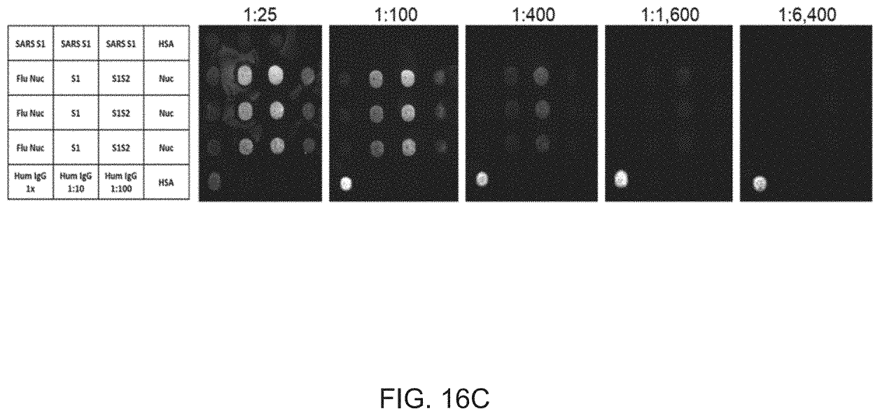

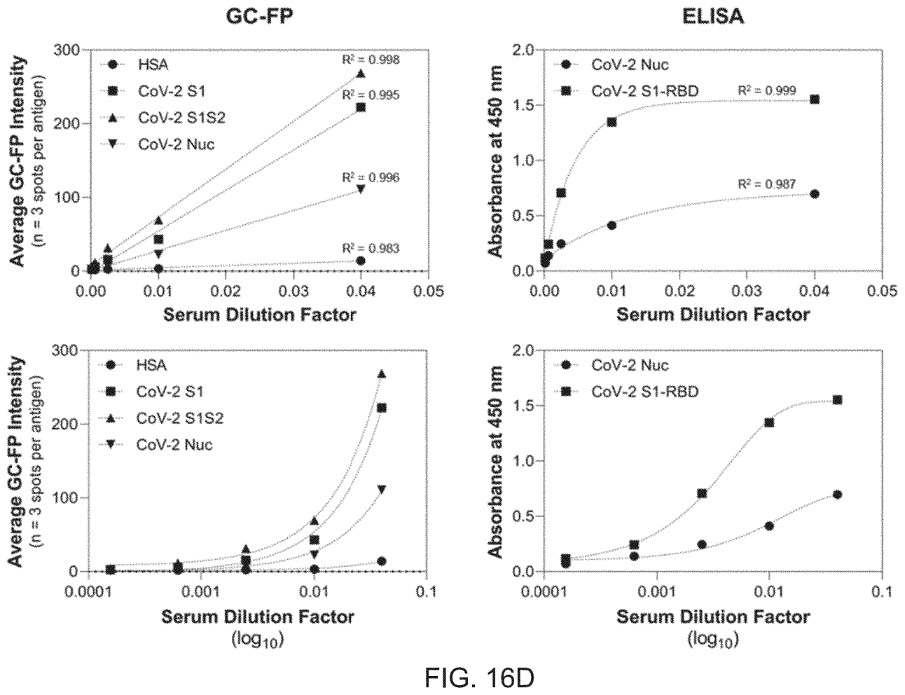

To assess GC-FP for quantitative determination of antibody concentration (titer), individual GC-FP chips were processed using dilutions of a COVID-19 positive serum sample received from the NYS Department of Health (sample DIL0526-3). Sample dilutions ranged from 1:25 to 1:25,600 and GC-FP testing results were compared to ELISA against S1 and Nuc, using commercial ELISA kits (Ray Biotech). The results of this experiment (FIGS. 16 A-C), demonstrate that GC-FP could detect IgG at a minimum dilution of 1:1,600 and that the commercial ELISA kit could detect IgG to a minimum dilution of 1:6,400. When GC-FP and ELISA data were plotted as a function of dilution factor (FIG. 16D) and fit with either linear regression (GC-FP) or partial least squares regression (ELISA), high goodness of fit (R-square >0.98) was observed for all antigens.

Recent COVID-19 antibody testing studies have shown that antibody titers in the range of 1:320 or higher could be considered eligible for convalescent plasma donation (for convalescent plasma therapy) (See Wajnberg, A., Amanat, F., Firpo, A., et al., 2020. medRxiv, 2020.2007.2014.20151126). As shown in this work, GC-FP can detect antibodies down to 1:1,600 titer, and thus has the necessary sensitivity for determining clinically and therapeutically relevant seroconversion status. The fact that GC-FP has a linear response for all antigens tested makes it easier to quantify antibody concentrations across the full dynamic range, and to directly compare a subject's response to different viral antigens.

FIGS. 16A-16D depict a quantitative comparison of GC-FP and ELISA for detection of IgG against multiple COVID-19 antigens. Sample DIL0526-3 was used for both GC-FP and ELISA testing, at dilutions ranging from 1:25 to 1:26,600 in PBS-T.

### Quantification of Antibody Titer and Comparison to ELISA

To assess GC-FP for quantitative determination of antibody concentration (titer), individual GC-FP chips were processed using dilutions of a COVID-19 positive serum sample received from the NYS Department of Health (sample DIL0526-3). Sample dilutions ranged from 1:25 to 1:25,600 and GC-FP testing results were compared to ELISA against S1 and Nuc, using commercial ELISA kits (Ray Biotech). The results of this experiment (FIG. 16A-C), demonstrate that GC-FP could detect IgG at a minimum dilution of 1:1,600 and that the commercial ELISA kit could detect IgG to a minimum dilution of 1:6,400. When GC-FP and ELISA data were plotted as a function of dilution factor (FIG. 16D) and fit with either linear regression (GC-FP) or partial least squares regression (ELISA), high goodness of fit (R-square >0.98) was observed for all antigens.

Recent COVID-19 antibody testing studies have shown that antibody titers in the range of 1:320 or higher could be considered eligible for convalescent plasma donation (for convalescent plasma therapy)(Wajnberg et al. 2020). As shown in this work, GC-FP can detect antibodies down to 1:1,600 titer, and thus has the necessary sensitivity for determining clinically and therapeutically relevant seroconversion status. The fact that GC-FP has a linear response for all antigens tested makes it easier to quantify antibody concentrations across the full dynamic range, and to directly compare a subject's response to different viral antigens.

### CONCLUSIONS

Rapid, accurate, and quantitative antibody tests are needed as part of the global response to the COVID-19 pandemic. In addition to epidemiological and seroconversion studies, such tests have the potential to confirm an individual's immunity status following prior infection or vaccination. In this work, it has been demonstrated that GC-FP can be used to simultaneously measure antibody levels for multiple, antigens in a single sample. The assay is quantitative across a large dynamic range (serum dilutions ranging from 1:25-1:1,600) and is highly correlated with gold-standard antibody detection tests, including ELISA and MIA (FIGS. 14 & 16A-D). Although GC-FP was not able to match ELISA for detecting antibodies in the lowest dilution (1:1,600 vs. 1:6,400), this may be overcome by modifying the GC-FP imaging system, including addition of a more sensitive camera. The GC-FP system could also include a point-of-care platform for on-site clinical diagnostics.

The entire assay time for performing antibody detection is below 30 min (27 min for all fluidic processing steps, and less than 1 min required for GC-FP fluorescence imaging), which is significantly shorter than either ELISA or MIA, which take 2-3 hrs to complete. Alternative antibody testing approaches, such as lateral flow assays, may be completed in <10 min, but suffer from low accuracy and lack of multiplexing (See Lisboa Bastos, M., Tavaziva, G., Abidi, S. K., et al., 2020. The BMJ 370, m2516; Sethuraman, N., Jeremiah, S. S., Ryo, A., 2020. JAMA 323(22), 2249-2251; and Younes, N., Al-Sadeq, D. W., Al-Jighefee, H., et al., 2020. Viruses 12(6), 582). This study also demonstrates that dried blood spots are a viable sample matrix for COVID-19 antibody testing. Using dried blood spots reduces the complexity of sample, collection, handling and storage versus venipuncture-based whole blood collection.

This work establishes the potential of GC-FP for detecting antibodies against COVID-19 antigens in both serum and dried blood spots. As more samples are tested with the GC-FP platform, the major goal will be to retain 100% selectivity while maximizing sensitivity. One way to achieve this will be to compare antibody responses to additional COVID-19 antigens, as shown with the addition of the RBD antigen. Further, GC-FP results should be compared to other tests, such as antibody neutralization, which provides a measure of whether an individual's antibodies can neutralize the virus to prevent infection. It is possible that maximizing sensitivity and specificity, as well as correlating antibody levels with neutralization testing will require knowledge of antibody levels against multiple antigens. Understanding antibody responses across various immunoglobulin classes may also be useful for determining the stage of an individual's seroconversion response, and could be useful when analyzing other bodily fluids, such as saliva (Randad et al. 2020). Thus, the multiplexed microarray format of the GC-FP antibody detection assay will have great utility in the future.

### Supplement to Example I

Positive samples were convalescent serum specimens received as part of the Wadsworth Center's Diagnostic Immunology Laboratory (DIL) testing program that were SARS-CoV-2 confirmed by RT-PCR and at least 21 days post symptom onset. Samples were pre-screened by the DIL using a multiplex MIA with both the CoV-2 nucleocapsid (Nuc) protein and the 2019 SARS CoV-2 receptor binding domain (RBD) on a Luminex detection platform (Yang, H. S., Racine-Brzostek, S. E., Lee, W. T., et al., 2020. Clinica Chimica Acta 509, 117-125). The specimens were defined as reactive if one or both of the antigens was reactive. To determine reactive vs. non-reactive, an MIA assay cut-off was determined using 94 normal serum samples (collected prior to 2019). Cut-off values were based on the mean of the medial fluorescence intensity (MFI) plus 6 standard deviations. Samples received from the DIL for GC-FP analysis were tested blind (no sample information provided). After GC-FP test results were obtained, DIL testing results were provided for the purpose of comparison. Blood serum was prepared after venipuncture and collection in serum collection tubes by centrifugation at 1,000 rpm for 15 min, followed by removal of the supernatant (serum). All serum samples were kept at −20° C. for short-term storage, or −80° C. for long-term storage.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed:

1. A method for a simultaneous qualitative and quantitative analysis of a plurality of biomarkers contained in a sample, comprising:
capturing one or more biomarkers on a substrate configured to bind a plurality of biomarkers to a plurality of binding sites, wherein at least two binding sites are configured to bind at least two biomarkers, and wherein when one or more biomarkers are present forming one or more bound biomarkers-of-interest;
contacting the one or more bound biomarkers-of-interest with one or more fluorescent binding partners to form one or more fluorescent complexes;
contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes;
detecting emission light of the one or more fluorescent complexes; and
determining a type and/or quantity of the plurality of biomarkers.

2. The method of claim 1, wherein the one or more biomarkers are immunoglobulins.

3. The method of claim 2, wherein the immunoglobulins comprise one or more of immunoglobulin G (IgG), immunoglobulin M (IgM), immunoglobulin A (IgA), or isotypes or combinations thereof.

4. The method of claim 1, wherein the one or more biomarkers are serum antibody targets.

5. The method of claim 1, wherein the one or more biomarkers are antigens comprising: BBA69, BBA70, BmpA, DbpA, DbpB, ErpL, OspC, OspD, P41, P58, VIsE, or combinations hereof.

6. The method of claim 1, wherein the substrate is disposed within a cell.

7. The method of claim 6, wherein the cell is a fluidic flow cell or a test-tube.

8. The method of claim 1, wherein each binding site of the plurality of binding sites comprises two or more predetermined antigens configured to bind to two or more different predetermined biomarkers-of-interest.

9. The method of claim 1, wherein the plurality of biomarkers are derived from blood serum, blood plasma, whole blood, dried blood.

10. The method of claim 1, wherein the type is characterized as immunoglobulin G (IgG), immunoglobulin M (IgM), immunoglobulin A (IgA), or isotypes or combinations thereof.

11. The method of claim 1, wherein the type is further characterized as virus specific to SARS-COV-2.

12. The method of claim 1, wherein the plurality of biomarkers are antibodies specific to a SARS-COV-2 full-length spike protein (SEQ ID NO:1), SARS-COV-2 spike 1 protein (SEQ ID NO: 2, SARS-COV-2 envelope protein (N) (SEQ ID NO: 3).

13. The method of claim 1, wherein contacting the substrate with a source of collimated, polarized light, further comprises positioning the substrate within a grating-coupled fluorescent plasmonic (GC-FP) detection platform.

14. The method of claim 1, wherein the substrate is further characterized as a GC-FP assay chip.

15. The method of claim 1, wherein the source of collimated polarized light is configured to cause fluorescent emission intensity greater than 10×, greater than 100×, greater than 500×, or greater than 1000×.

16. The method of claim 1, wherein detecting emission light of the one or more fluorescent complexes further comprises forming a fluorescent image on an antigen array or map of antigens upon the substrate.

17. The method of claim 1, wherein the substrate is disposed atop a dip-stick configured to be deposited into a cell characterized as a tube.

18. An apparatus for qualitative and quantitative analysis of a plurality of biomarkers contained in a sample, comprising:
a substrate comprising a plurality of binding sites, wherein two or more of the plurality of binding sites comprises two or more predetermined antigens configured to bind to two or more predetermined biomarkers-of-interest and form two or more bound biomarkers-of-interest when biomarkers-of-interest are flowed over the substrate, and wherein the biomarkers-of-interest, when present, are configured to bind one or more fluorescent binding partners to form one or more fluorescent complexes; and
a grating-coupled fluorescent plasmonic (GC-FP) detection platform, wherein the platform is configured for contacting the substrate with a source of collimated, polarized light, wherein the source of collimated polarized light is configured to emit light at a predetermined wavelength appropriate for transferring energy to a plurality of surface plasmons and excite fluorescence of the one or more fluorescent complexes.

19. The apparatus of claim 18, wherein the substrate is disposed within a cell, or disposed atop a dip-stick and configured to fit within the cell, and wherein the cell is a test-tube.

20. The apparatus of claim 18, wherein the GC-FP detection platform comprises a detector configured to detect emission light from one or more fluorescent complexes when one or more biomarkers-of-interest are contacted with a plurality of bonding sites.

21. The apparatus of claim 18, wherein the substrate comprises a plurality of binding sites, wherein each binding site of the plurality of binding sites comprises two or more predetermined antigens configured to bind to two or more predetermined antibodies-of-interest.

22. The apparatus of claim 18, wherein the plurality of biomarkers are serum derived antibodies, antibodies from whole blood, or antibodies from dried blood.
